(19) United States
(12) Patent Application Publication
(10) Pub. No.: US 2012/0234326 A1
(43) Pub. Date: Sep. 20, 2012

(54) MASK SYSTEM

(75) Inventors: Damien Julian MAZZONE, Concord West (AU); Justin John Formica, Voyager Point (AU); Grant Moiler, Chipping Norton (AU); Kai Stuebiger, North Sydney (AU); Sofietje Pellegatti, Turramurra (AU)

(73) Assignee: ResMed Limited, Bella Vista (AU)

(21) Appl. No.: 13/350,622

(22) Filed: Jan. 13, 2012

Related U.S. Application Data
(60) Provisional application No. 61/457,143, filed on Jan. 14, 2011.

(51) Int. Cl. A61M 16/06

(52) U.S. Cl. 128/206.26; 128/206.21; 128/206.24

(57) ABSTRACT

A mask system includes a frame and a cushion provided to the frame. The frame provides a brace or fulcrum like structure about which the cushion may pivot, bend, and/or flex.

### CROSS REFERENCE TO RELATED APPLICATIONS

This application claims priority to U.S. Provisional Application Ser. No. 61/457,143, filed Jan. 14, 2011, which is incorporated herein by reference in its entirety.

### FIELD OF TECHNOLOGY

The present technology relates to a mask system used for treatment, e.g., of Sleep Disordered Breathing (SDB) with Continuous Positive Airway Pressure (CPAP) or Non-Invasive Positive Pressure Ventilation (NIPPV).

### BACKGROUND OF TECHNOLOGY

Patient interfaces, such as a full-face or nasal mask systems, for use with blowers and flow generators in the treatment of Sleep Disordered Breathing (SDB), typically include a soft face-contacting portion, such as a cushion, and a rigid or semi-rigid shell or frame. In use, the interface is held in a sealing position by headgear so as to enable a supply of air at positive pressure (e.g., 2-30 cm H₂O) to be delivered to the patient's airways.

One factor in the efficacy of therapy and compliance of patients with therapy is the comfort and fit of the patient interface.

The present technology provides alternative arrangements of mask systems to enhance the efficacy of therapy and compliance of patients with therapy.

### SUMMARY OF TECHNOLOGY

One aspect of the disclosed technology relates to a mask.

Another aspect of the disclosed technology relates to a mask having a cushion.

Another aspect of the disclosed technology relates to a mask having a cushion including a pocket or bladder filled with a conformable material.

Another aspect of the disclosed technology relates to a mask having a cushion adapted to form a seal with a patient's face and including a first region adapted to form a seal with the patient's face by a tensile force and a second region adapted to form a seal with the patient's face by a compression force.

Another aspect of the disclosed technology relates to a mask having a cushion adapted to form a seal with a patient's face and including a first region adapted to form a seal with the patient's face by a tensile force and a second region adapted to form a seal with the patient's face by a compression force. The tensile force may be provided by a membrane and an undercushion of the cushion, and the compression force may be provided by a bladder filled with a conformable material.

Another aspect of the disclosed technology relates to a cushion including a bladder having an overflow region.

Another aspect of the disclosed technology relates to a cushion including a bladder filled with a conformable material. The bladder may have an overflow region adapted to receive a portion of the conformable material that exceeds a maximum fill level of the bladder.

Another aspect of the disclosed technology relates to a cushion including a bladder filled with a conformable material. The bladder may have an overflow region constructed and arranged to ensure a maximum fill level of the bladder is constant.

Another aspect of the disclosed technology relates to a cushion including a bladder filled with a conformable material. The bladder may have an overflow region constructed and arranged to ensure a maximum fill level of the bladder is constant thereby enabling a rigid component (e.g., a frame) to interface with the filled bladder at the same location on all cushions.

Another aspect of the disclosed technology relates to a cushion including a bladder arranged to buckle or pivot about a rigid component (e.g., a frame) in use to prevent the cushion from bottoming out on the rigid component.

Another aspect of the disclosed technology relates to a cushion including a bladder arranged to buckle or pivot about a rigid component (e.g., a frame) in use to prevent the cushion from bottoming out on the rigid component. The geometry of the cushion may be arranged to encourage the bladder to buckle or pivot in a predetermined direction.

Another aspect of the disclosed technology relates to a method of facilitating a molding process of a cushion, wherein the cushion design includes one or more features which facilitate the molding process.

Another aspect of the disclosed technology relates to a method of facilitating a molding process of a cushion, wherein the cushion design includes one or more features which facilitate the molding process, and the molding process involves a multiple cavity tool.

Another aspect of the disclosed technology relates to a method of facilitating a molding process of a cushion, wherein the cushion design includes one or more features which facilitate the molding process. In an example, the cushion design may include a single line of draw.

Another aspect of the disclosed technology relates to a mask system including a frame and a cushion provided to the frame. The frame provides a brace or fulcrum like structure about which the cushion may pivot, bend, and/or flex.

Another aspect of the disclosed technology relates to a mask system including a fillable bladder filled with or containing a gel or super soft solid. The fillable bladder may vary in depth along the sides of the cushion from the nose region toward the cheek region.

Another aspect of the disclosed technology relates to a mask system including a strap connector having a hook, the hook including a retaining portion for allowing user-friendly insertion and retainment of straps.

Another aspect of the disclosed technology relates to a mask system including a strap connector having a hook, the hook including a retaining portion and a depending arm for allowing user-friendly insertion and retainment of straps.

Another aspect of the disclosed technology relates to a mask system including a strap connector having a hook with a retaining portion for allowing user-friendly insertion and retainment of straps. The hook includes an inclined slot for inserting a strap.

Another aspect of the disclosed technology relates to a mask system including a strap connector having a hook with a retaining portion for allowing user-friendly insertion and retainment of straps. The hook includes a curved slot for inserting a strap.

Another aspect of the disclosed technology relates to a sealing arrangement for a mask system including a cushion molded in a single piece from an elastic material. The cushion includes a sealing membrane adapted to form a seal with the patient's face, an undercushion, and a fillable bladder filled with or containing a gel or super soft solid. The undercushion and fillable bladder are provided in one or more selected regions of the cushion. The undercushion may be removed from some regions of the cushion, e.g., there may be no undercushion provided in the nasal bridge region, the upper lip region, etc. The fillable bladder may be provided in the lower cheek and corner of nose regions of the cushion.

Another aspect of the disclosed technology relates to a mask system including a frame including a generally open construction and a cushion including a breathing chamber and adapted to form a seal with the patient's face. The cushion is supported by the frame such that an exterior surface of the cushion engages an interior surface of the frame. One or more portions of the cushion include a fillable bladder filled with gel or super soft solid.

Another aspect of the disclosed technology relates to a cushion for a mask system including a sealing membrane adapted to form a seal with the patient's face, a support layer, e.g., a fillable bladder filled with a gel or super soft solid, to support the sealing membrane, and a composite sealing portion including the sealing membrane and at least a portion of the support layer. The membrane is removed in at least one region of the cushion, e.g., the corner of nose region, such that the support layer is adapted to seal directly on the patient's face where the membrane is not present. The composite sealing portion may include a membrane part and a non-membrane part. The non-membrane part may be the support layer in which case the support layer has a dual function to support the membrane in at least one region, and to form a seal structure for sealing with the patient's skin. The membrane part and the support layer may include a transition region, or they may be substantially co-planar or otherwise flush or substantially seamless.

Another aspect of the disclosed technology relates to a cushion for a mask system including a sealing membrane and a fillable bladder filled with or containing a gel or super soft solid, wherein the fillable bladder is provided in one or more selected regions of the cushion. For example, the bladder may be provided around the entire perimeter of the cushion, the bladder may only be provided in the side of nose and cheek regions of the cushion, the bladder may only be provided in the corner of nose region of the cushion, the bladder may be configured such that more bladder is provided in the corner of nose region than the side of the nose regions, or the bladder may be configured such that more bladder is provided in the nasal bridge region so that the cushion can fit a range of nose bridge heights.

Another aspect of the disclosed technology relates to a mask system including a frame and a cushion provided to the frame. The frame interfaces with the cushion to separate a functional or active portion of the cushion or bladder from a tactile or aesthetic portion of the cushion or bladder. The frame may form a brace or fulcrum of sorts about which a portion of the cushion may bend, flex, and/or pivot

Another aspect of the disclosed technology relates to a mask system including a frame and a cushion provided to the frame. The frame may include side engagement tabs that extend from the main body of the frame and are positioned to interface and support the cushion. In an example, the tabs may support the cushion so that it is supported in sealing engagement with the sides of the patient's nose or cheek region. Also, the tabs may support the cushion to prevent collapse or blowout of the cushion away from the patient's face in use.

Another aspect of the disclosed technology relates to a mask system including a frame and a cushion provided to the frame. The frame may assist in retaining and positioning the cushion as well as capping a fillable bladder of the cushion. For example, the bladder may include one or more openings along the non-face-contacting side of the cushion, and the frame may abut these one or more openings to close it off. In an example, the frame may have one or more lugs or protrusions adapted to engage with respective openings of the bladder, which may further aid alignment of the cushion with respect to the frame.

Other aspects, features, and advantages of this technology will become apparent from the following detailed description when taken in conjunction with the accompanying drawings, which are a part of this disclosure and which illustrate, by way of example, principles of this technology.

### BRIEF DESCRIPTION OF THE DRAWINGS

The accompanying drawings facilitate an understanding of the various examples of this technology. In such drawings:

FIG. 1-1 is a three-dimensional representation of a cushion for a nasal mask system according to an example of the disclosed technology;

FIG. 1-2 is a front view of the cushion of FIG. 1-1;

FIG. 1-3 is a side view of the cushion of FIG. 1-1;

FIG. 1-4 is a bottom view of the cushion of FIG. 1-1;

FIG. 1-5 is a top view of the cushion of FIG. 1-1;

FIG. 1-6 is a cross-section view through line 1-6-1-6 of FIG. 1-5;

FIG. 1-7 is a cross-section view through line 1-7-1-7 of FIG. 1-2;

FIG. 1-8 is an enlarged portion of the cross-section of FIG. 1-7;

FIG. 1-9 is a cross-section view through a nasal bridge region of the cushion of FIG. 1-1;

FIG. 1-10 is a cross-section view through a side of nose region of the cushion of FIG. 1-1;

FIG. 1-11 is a cross-section view through an upper cheek region of the cushion of FIG. 1-1;

FIG. 1-12 is a cross-section view through a lower cheek region of the cushion of FIG. 1-1;

FIG. 1-13 is a cross-section view through a corner of nose region of the cushion of FIG. 1-1;

FIG. 1-14 is a cross-section view through an upper lip region of the cushion of FIG. 1-1;

FIG. 1-15 is another cross-section view of the cushion of FIG. 1-1;

FIGS. 2-1 and 2-2 show bladders according to alternative examples of the disclosed technology;

FIG. 3-1 shows a bladder wall configuration according to an example of the disclosed technology;

FIG. 3-2 shows a bladder wall configuration according to another example of the disclosed technology;

FIG. 3-3 shows a bladder wall configuration according to another example of the disclosed technology;

FIG. 3-4 shows a bladder wall configuration according to another example of the disclosed technology, and showing deflection of the bladder wall in use;

FIGS. 3-5(1) and 3-5(2) show a bladder configuration according to another example of the disclosed technology, and showing deflection of the bladder in use;

FIG. 4 shows a cushion according to another example of the disclosed technology;

FIGS. 5-1 and 5-2 show a cushion attached to a frame according to an example of the disclosed technology;

FIG. 6-1 is a front view of the cushion according to another example of the disclosed technology;

FIG. 6-2 is a cross-section view through a nasal bridge region of the cushion of FIG. 6-1;

FIG. 6-3 is a cross-section view through a side of nose region of the cushion of FIG. 6-1;

FIG. 6-4 is a cross-section view through an upper cheek region of the cushion of FIG. 6-1;

FIG. 6-5 is a cross-section view through a lower cheek region of the cushion of FIG. 6-1;

FIG. 6-6 is a cross-section view through a corner of nose region of the cushion of FIG. 6-1;

FIG. 6-7 is a cross-section view through an upper lip region of the cushion of FIG. 6-1;

FIG. 7 is a perspective view of a mask system including a frame and cushion according to an example of the disclosed technology;

FIG. 8-1 is a front perspective view of a frame according to an example of the disclosed technology;

FIG. 8-2 is a side view of the frame of FIG. 8-1;

FIG. 8-3 is a rear view of the frame of FIG. 8-1;

FIG. 8-4 is a rear perspective view of the frame of FIG. 8-1;

FIGS. 8-5 and 8-6 show engagement of the frame of FIG. 8-1 with a cushion according to an example of the disclosed technology;

FIG. 9-1 is a front perspective view of a frame according to an example of the disclosed technology;

FIGS. 9-2 to 9-4 show engagement of the frame of FIG. 9-1 with a cushion according to an example of the disclosed technology, and support by headgear on the patient's head;

FIG. 10 shows a cross-section through a side of nose region of a cushion according to an example of the disclosed technology;

FIG. 11 shows a cross-section through a corner of nose region of a cushion according to an example of the disclosed technology;

FIG. 12 shows a cross-section through an upper lip region of a cushion according to an example of the disclosed technology;

FIG. 13 shows a cross-section through a cheek region of a cushion according to an example of the disclosed technology;

FIG. 14 shows a cross-section through a nasal bridge region of a cushion according to an example of the disclosed technology;

FIG. 15 shows a mask system including a cushion according to an example of the disclosed technology;

FIGS. 16-1 to 16-6 show cushions with bladder locations according to alternative examples of the disclosed technology;

FIG. 17 shows a cushion including a bladder according to an example of the disclosed technology; and

FIGS. 18 and 19 show a mask system with a headgear arrangement according to an example of the disclosed technology;

FIG. 20 shows a mask system with a headgear arrangement according to an example of the disclosed technology; and

FIGS. 21 and 22 show a mask system with a headgear arrangement according to an example of the disclosed technology;

FIG. 23-1 is a three-dimensional representation of a cushion according to another example of the disclosed technology;

FIG. 23-2 is a front view of the cushion of FIG. 23-1;

FIG. 23-3 is a back view of the cushion of FIG. 23-1;

FIG. 23-4 is a top view of the cushion of FIG. 23-1;

FIG. 23-5 is a bottom view of the cushion of FIG. 23-1;

FIG. 23-6 is a side view of the cushion of FIG. 23-1;

FIG. 23-7 is another front view of the cushion of FIG. 23-1;

FIG. 23-8 is a cross-section view through line 23-8-23-8 of FIG. 23-7;

FIG. 23-9 is a cross-section view through line 23-9-23-9 of FIG. 23-7;

FIG. 23-10 is a cross-section view through line 23-10-23-10 of FIG. 23-7;

FIG. 23-11 is a cross-section view through line 23-11-23-11 of FIG. 23-7;

FIG. 23-12 is a cross-section view through line 23-12-23-12 of FIG. 23-7;

FIG. 23-13 is a cross-section view through line 23-13-23-13 of FIG. 23-7;

FIG. 23-14 is a cross-section view through line 23-14-23-14 of FIG. 23-7;

FIG. 24-1 is a three-dimensional representation of a cushion according to another example of the disclosed technology;

FIG. 24-2 is a front view of the cushion of FIG. 24-1;

FIG. 24-3 is a back view of the cushion of FIG. 24-1;

FIG. 24-4 is a top view of the cushion of FIG. 24-1;

FIG. 24-5 is a bottom view of the cushion of FIG. 24-1;

FIG. 24-6 is a side view of the cushion of FIG. 24-1;

FIG. 24-7 is another front view of the cushion of FIG. 24-1;

FIG. 24-8 is a cross-section view through line 24-8-24-8 of FIG. 24-7;

FIG. 24-9 is a cross-section view through line 24-9-24-9 of FIG. 24-7;

FIG. 24-10 is a cross-section view through line 24-10-24-10 of FIG. 24-7;

FIG. 24-11 is a cross-section view through line 24-11-24-11 of FIG. 24-7;

FIG. 24-12 is a cross-section view through line 24-12-24-12 of FIG. 24-7;

FIG. 24-13 is a cross-section view through line 24-13-24-13 of FIG. 24-7

FIG. 24-14 is a cross-section view through line 24-14-24-14 of FIG. 24-7;

FIG. 25-1 is a bottom view of a cushion according to another example of the disclosed technology;

FIG. 25-2 is a cross section view of the cushion of FIG. 25-1 along a line similar to the line 24-8-24-8 of FIG. 24-;

FIG. 25-3 is a front view of the cushion of FIG. 25-1;

FIG. 26 is a front perspective view of a frame according to an example of the disclosed technology;

FIG. 27 is a partial front perspective view of a frame according to an example of the disclosed technology;

FIGS. 28-1 to 28-18 show hooks according to examples of the disclosed technology;

FIG. 29 is a cross-section view of a cushion according to another example of the disclosed technology showing a bladder overflow region of the cushion.

### DETAILED DESCRIPTION OF ILLUSTRATED EXAMPLES

The following description is provided in relation to several examples (most of which are illustrated, some of which may not) which may share common characteristics and features. It is to be understood that one or more features of any one example may be combinable with one or more features of the other examples. In addition, any single feature or combination of features in any of the examples may constitute additional examples.

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

The term “air” will be taken to include breathable gases, for example air with supplemental oxygen.

### 1. Nasal Mask System

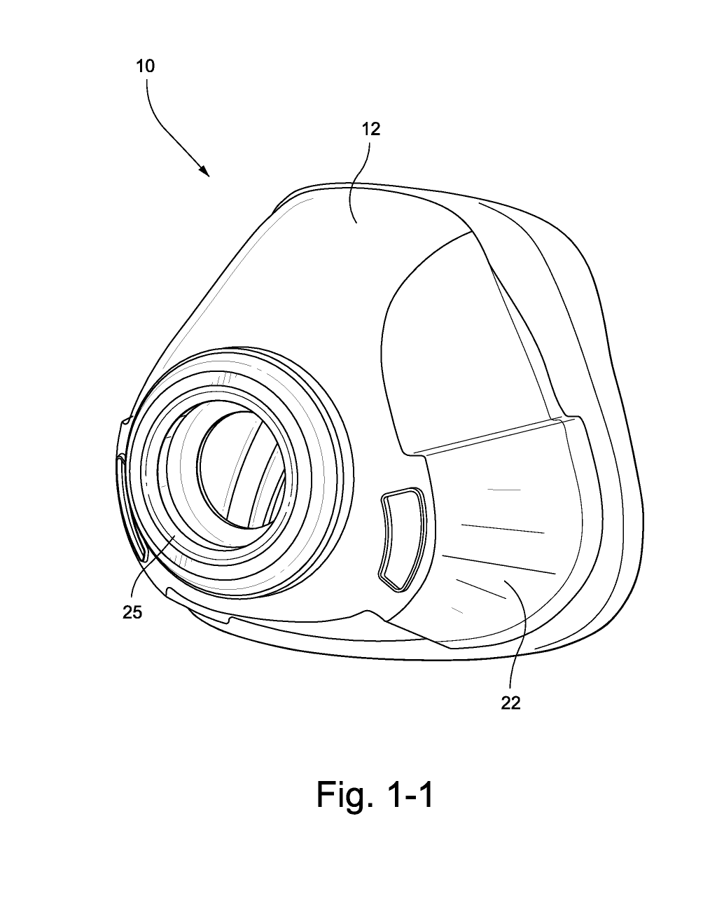

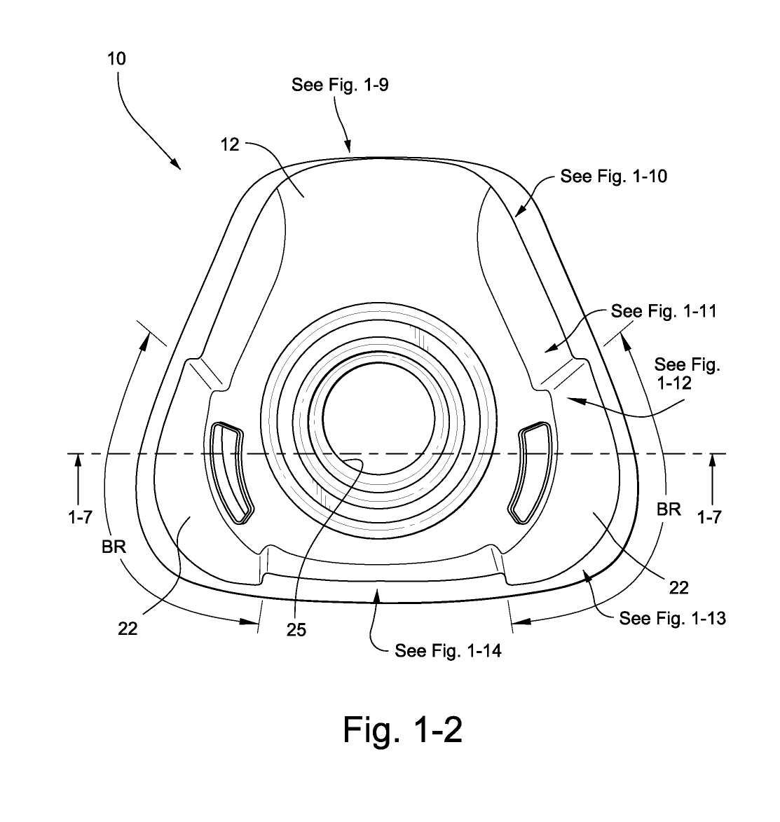

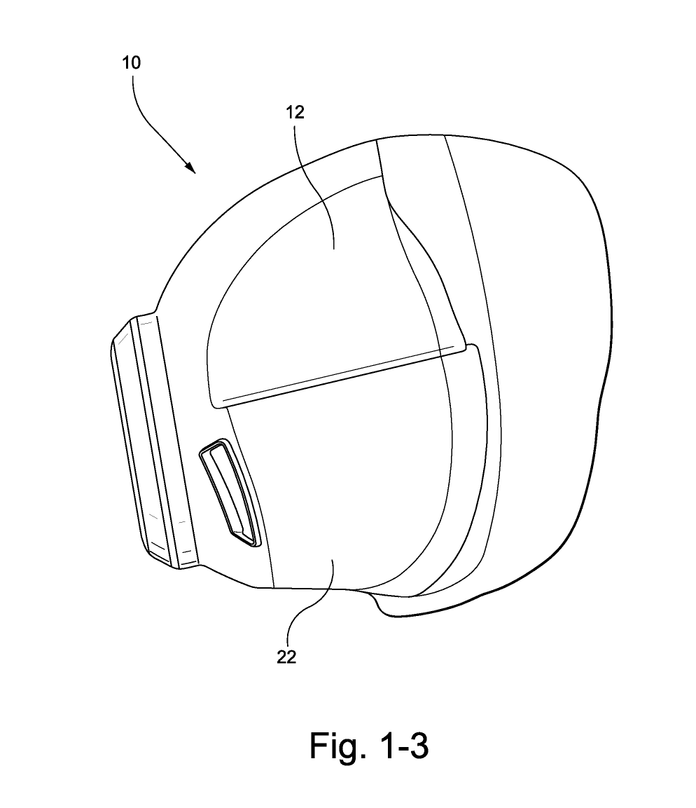

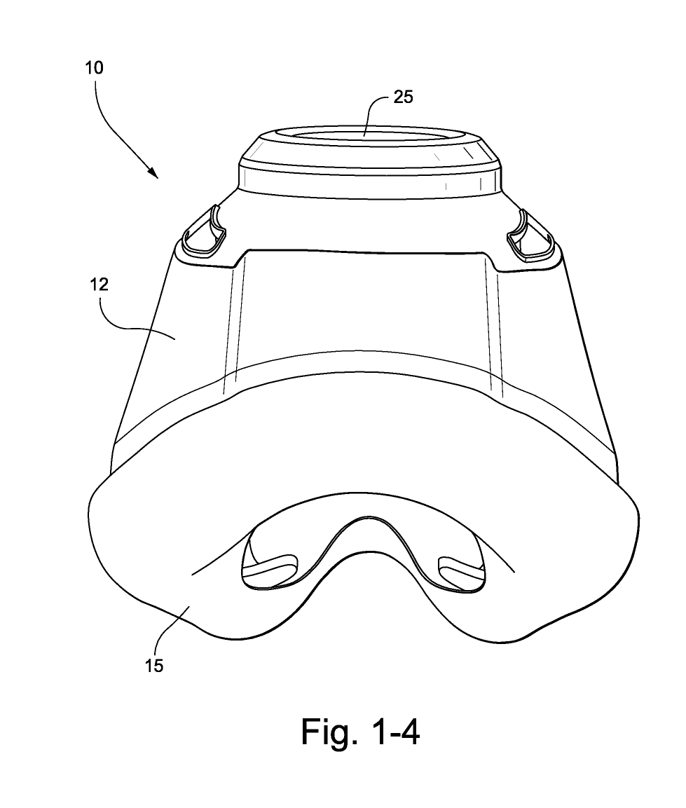

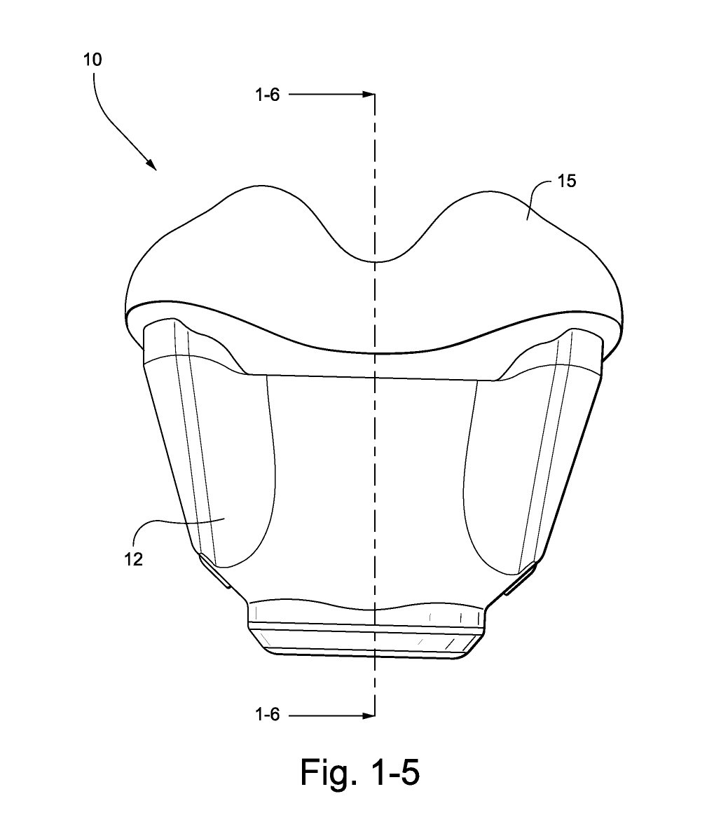

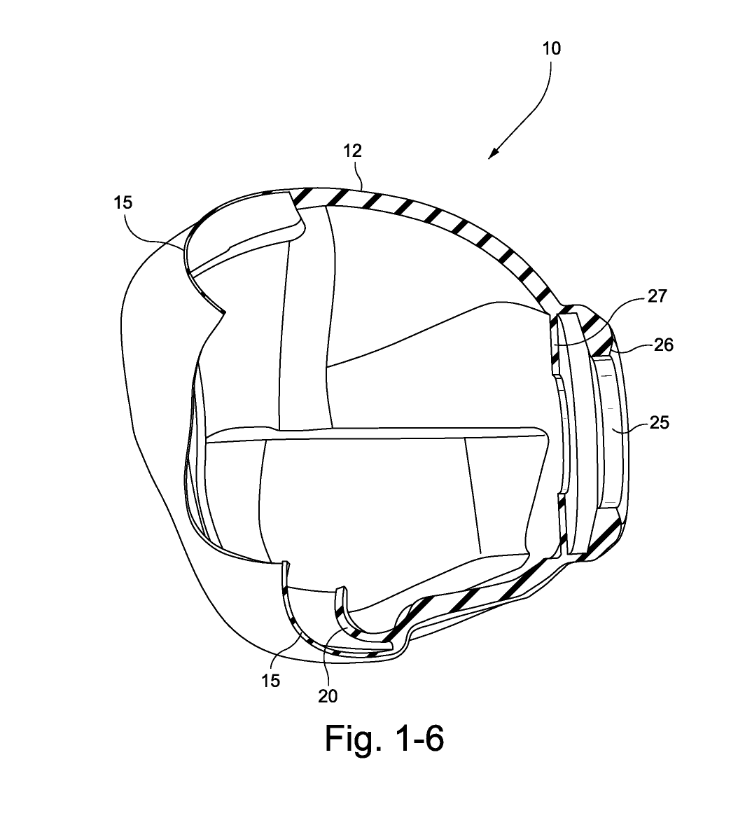

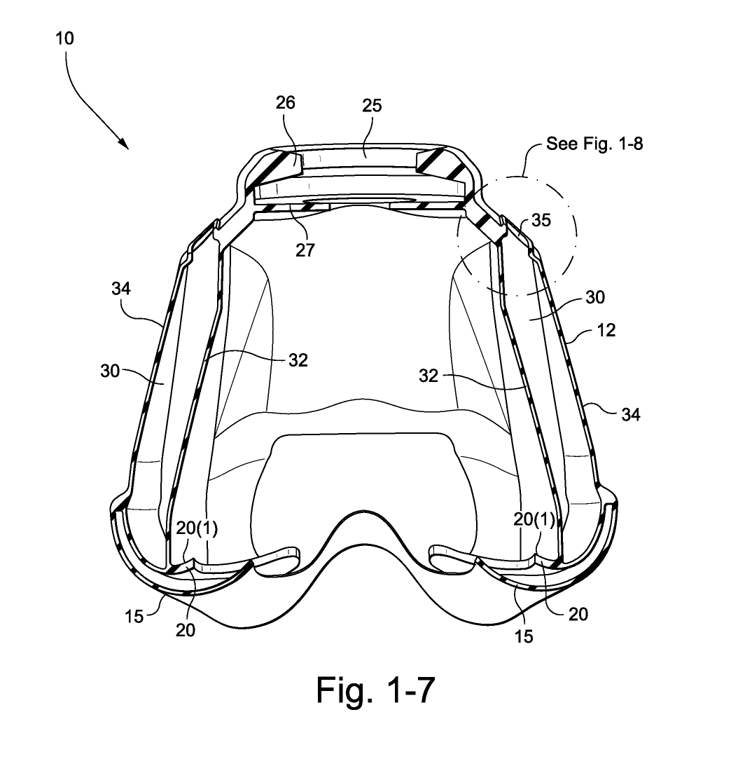

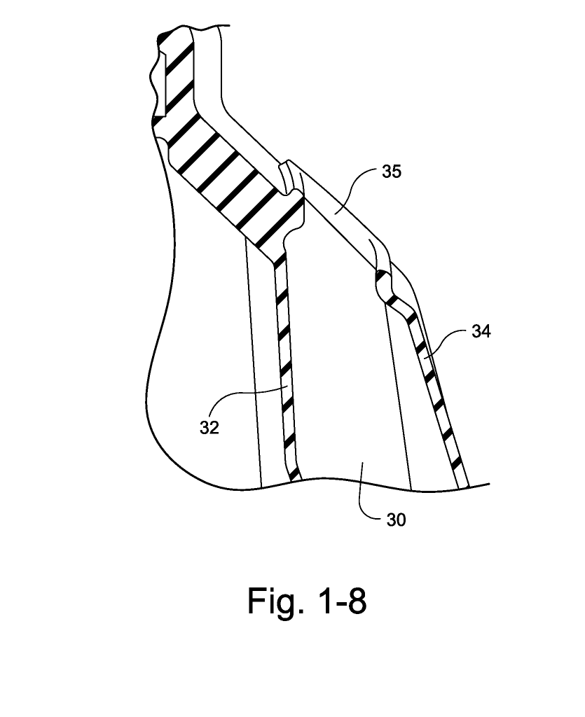

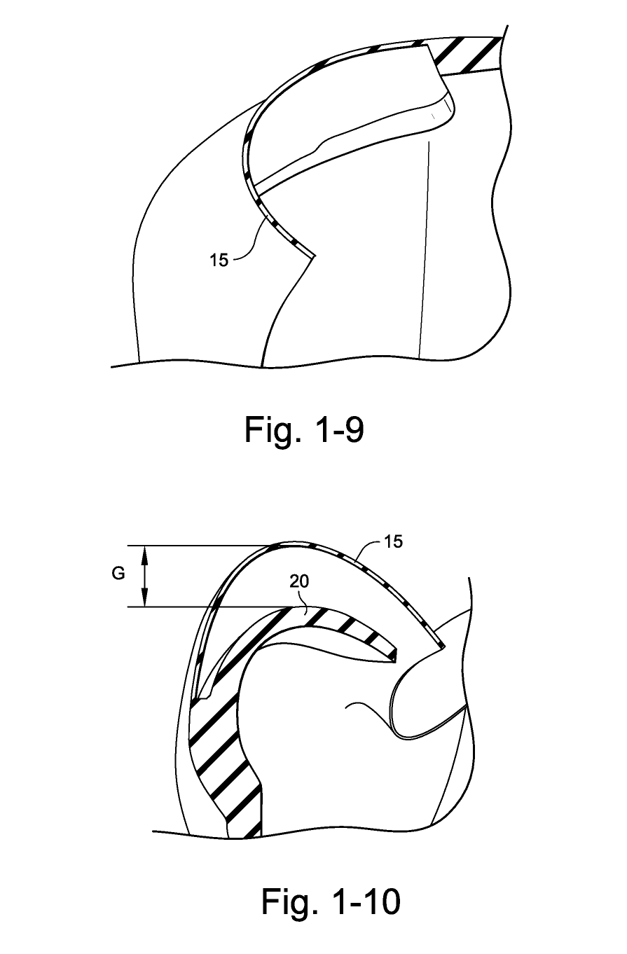

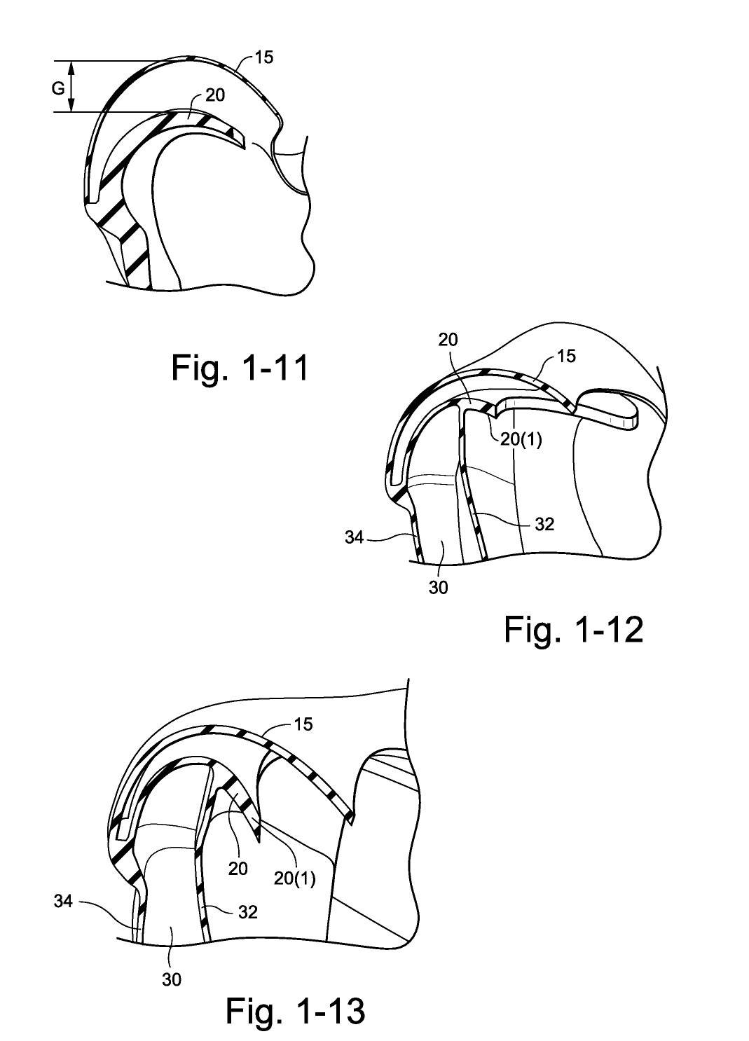

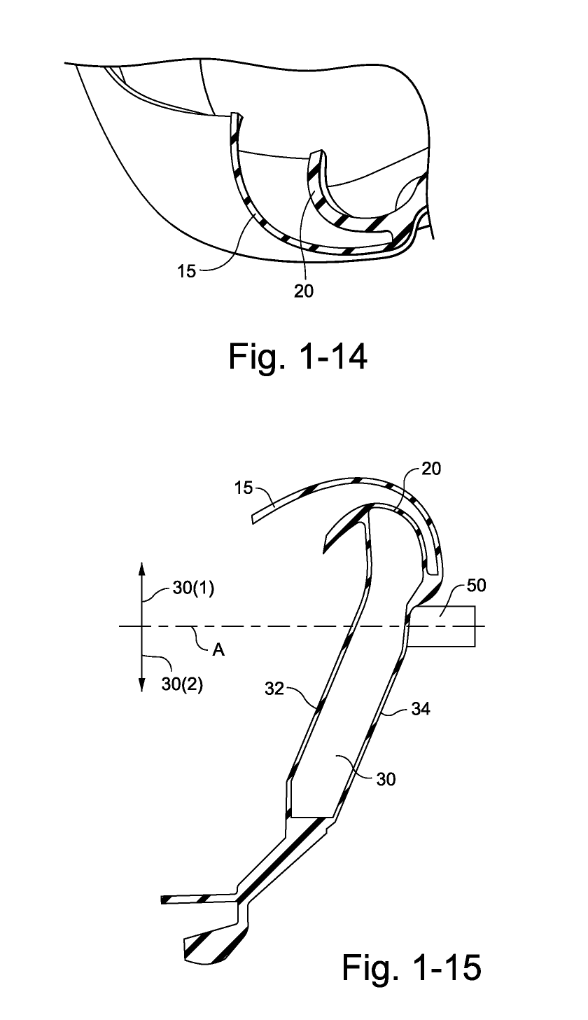

FIGS. 1-1 to 1-14 show a sealing arrangement 10 (e.g., cushion) for a nasal mask system according to an example of the disclosed technology. As described in greater detail below, the sealing arrangement includes a soft fillable material (e.g., gel, super soft solid (e.g., such as gel in their super soft state), gel-like material, or super soft solid-like material) in selected regions (e.g., along the patient's cheek and corners of the nose) of the sealing arrangement to enhance comfort, support, fit range, and/or reduce cost.

The sealing arrangement is structured to interface with a frame and form a seal with the patient's airway in use. The frame may be removably attached to headgear to maintain the nasal mask system in a desired adjusted position on the patient's face. Also, the sealing arrangement is structured to support an elbow adapted to be connected to an air delivery tube that delivers breathable gas to the patient. A swivel ring may be optionally provided to couple the elbow to the sealing arrangement. The nasal mask system is intended for use in positive pressure therapy for users with Obstructive Sleep Apnea (OSA), Central Sleep Apnea (CSA), or another respiratory disorder.

Exemplary frame arrangements, elbow arrangements, and interfacing arrangements for attaching the sealing arrangement to a frame and elbow are disclosed in U.S. Provisional Application No. 61/263,175, filed Nov. 20, 2009, which is incorporated herein by reference in its entirety. Also, exemplary fillable materials, tillable material properties, and fillable material arrangements are disclosed in WO/2009/062265 and WO 2009/143586, each of which is incorporated herein by reference in its entirety.

The sealing arrangement may be provided in a single size (e.g., one size fits most), or may be provided in two or more sizes (e.g., two size options). Also, the sealing arrangement may be modular for use in a modular-type mask system, i.e., the sealing arrangement may be interchangeable with another sealing arrangement and fit the same frame and/or elbow. The sealing arrangement may be structured so that it may be used or retrofit with existing mask systems.

In addition, the sealing arrangement may be suitable for multi patient multi use (MPMU), and provides reduced complexity for ease of use compared to existing gel or super soft solid masks.

While each example below is described as including a nasal interface type adapted to form a seal with the patient's airway, aspects of the technology may be adapted for use with other suitable interface types, e.g., full-face interface, etc.

### 2. Sealing Arrangement

The sealing arrangement provides a nasal interface adapted to engage the patient's face generally along nasal bridge, cheek, and upper lip regions of the patient's face.

Preferably, the sealing arrangement may be constructed and arranged in such a way so as to permit cost-effective molding of the sealing arrangement in a multiple cavity tool. In an example, the sealing arrangement is designed such that there is a single line of draw. Alternatively, in an arrangement having more than one line of draw and where sliders are required, the sliders may be designed to travel only a short distance to enable use of a reasonable size and therefore cost effective multiple cavity tool.

The sealing arrangement may be constructed and arranged to provide a portion of the cushion for the patient to touch that may be non-functional (i.e. the tactile portion of the cushion may provide limited to no sealing force). Such an arrangement may be desirable to increase patient engagement with the sealing arrangement without compromising the functionality of the product.

In an example, the sealing arrangement may be small and unobtrusive.

As shown in FIGS. 1-1 to 1-14, the sealing arrangement 10 includes a cushion 12 molded (e.g., injection molded) in a single piece from an elastic material such as silicone (e.g., liquid silicone rubber (LSR)). However, the cushion may be constructed of other suitable, generally flexible materials. The cushion defines a breathing chamber or cavity adapted to receive the patient's nose and provide air communication to the patient.

The sealing arrangement 10 may be formed by other suitable manufacturing methods, e.g., such as compression molding, machining, etc.

The face-contacting side of the cushion includes a dual-wall configuration in selected regions of the cushion. Specifically, as shown in FIGS. 1-2 and 1-9 to 1-14, the cushion 12 includes a single wall configuration, i.e., membrane 15 only, in the nasal bridge region, and the cushion includes a dual-wall configuration, i.e., membrane 15 and undercushion 20, in the remaining regions of the cushion, i.e., side of nose, upper and lower cheek, corner of nose, and upper lip regions, although the undercushion need not extend across the upper lip region. The membrane at least partially covers the undercushion and is generally softer and less stiff than the undercushion to provide a seal against the patient's face in use.

FIG. 1-9 shows a cross-section through the nasal bridge region of the cushion. As illustrated, the membrane 15 is substantially C-shaped, curved or arcuate. The membrane may be 0.1-0.7 mm thick. Preferably, the membrane is approximately 0.3 mm thick. The absence of undercushion 20 at the nasal bridge region may provide greater freedom and flexibility of the membrane 15 to accommodate and adapt to seal on a variety of patient noses. This arrangement may also result in lower force exerted by the mask onto the patient's nasal bridge as there is no undercushion to increase pressure and/or force in this region.

FIG. 1-10 shows a cross-section through the side of nose region of the cushion. Undercushion 20 is positioned substantially underneath membrane 15. Undercushion 20 may be structured to, support membrane 15 such that membrane 15 is adapted to form a seal with the patient. Undercushion 20 may have a thickness that is greater than the thickness of membrane 15. Preferably, in this region, the cushion may roll or exert a force lateral to the patient's nose so as to seal on the sides of the nose. The membrane 15 is intended to extend further into the breathing chamber than the undercushion 20. As a result, the patient's nose is likely to first contact the membrane 15 before contacting the undercushion 20. In addition, the gap G between the membrane 15 and the undercushion 20 may cause the patient's nose to first engage the membrane 15 rather than the undercushion 20. This arrangement permits the membrane 15 to be compliant and therefore the membrane can adapt to different nose shapes, such as high nose bridges and flat nose bridges. The undercushion 20 may then support the membrane 15 once the membrane has adapted to the shape of the patient's face.

FIG. 1-11 shows a cross-section through the upper cheek region of the cushion. In this region, the force exerted by the cushion should be normal to the patient's face. Since there may be less anthropometric variation across the population in this region, the gap G between the membrane 15 and the undercushion 20 is less. The radius of curvature of the undercushion 20 and membrane 15 is approximately the same.

FIG. 1-12 shows a cross-section through the lower cheek region of the cushion. In this region, there is a gel pocket 30 adjacent or connected to undercushion 20. Gel pocket 30 has an inner wall 32 and an outer wall 34. Outer wall 34 may be positioned inwards of membrane 15. Undercushion 20 is positioned inwardly and towards the patient's nose from the gel pocket 30. This is to encourage the gel pocket 30 to collapse inwards, thereby maintaining contact with the patient's skin and sealing.

FIG. 1-13 shows a cross-section through the corner of nose region of the cushion. This region is similar to the lower cheek region as shown in FIG. 1-12, however the undercushion 20 is longer. This is to ensure that the membrane 15 is supported so that it can take up the geometry of the patient's nose corner crease.

FIG. 1-14 shows a cross-section through the upper lip region of the cushion. Preferably, the undercushion 20 is small to non-existent or provides a lesser force compared to other regions of the undercushion 20 to prevent undue force being exerted on the patient's top lip, since this area can be sensitive.

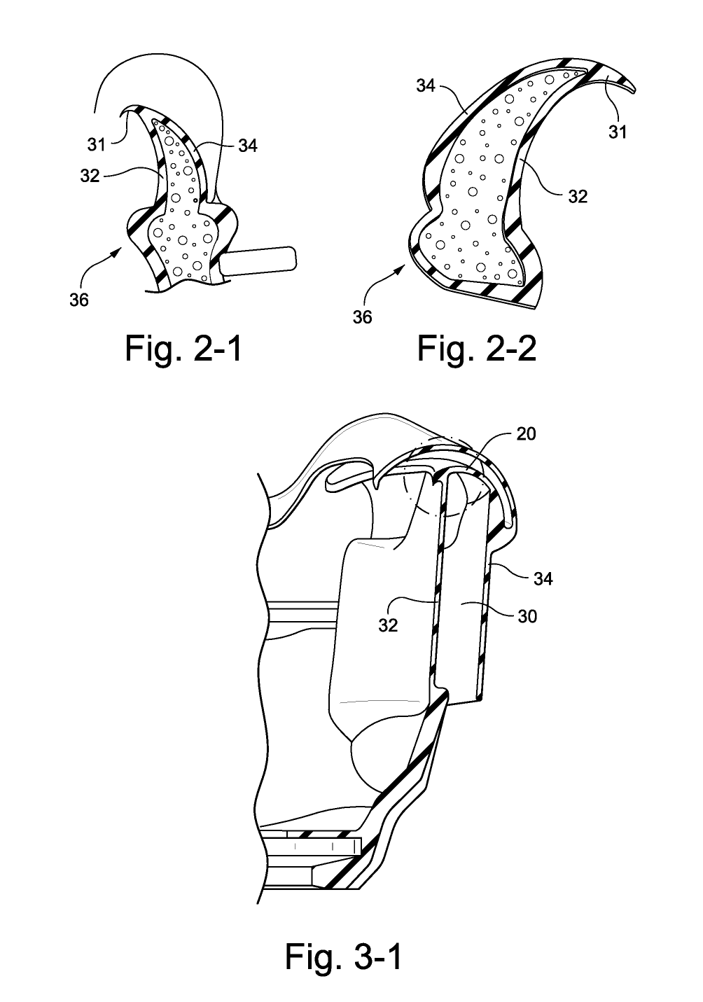

The cushion also includes a bladder, pocket, or chamber 30 in one or more selected regions of the cushion that is filled with a fillable material (e.g., gel, super soft solid, gel-like material, or super soft solid-like material). In the illustrated embodiment, as best shown in FIGS. 1-2, 1-7, 1-12, and 1-13, the bladder is only provided in the lower cheek and corner of nose regions of the cushion, e.g., see bladder region BR of cushion 10 in FIG. 1-2. The bladder may be provided in other regions, such as the nasal bridge, sides of the nose, top lip region, etc.

In use, the membrane defines a seal forming structure that is structured to engage and provide a seal to the patient's face, and the gel or super soft solid bladder defines a cushioning structure to add support and stability to the seal forming structure.

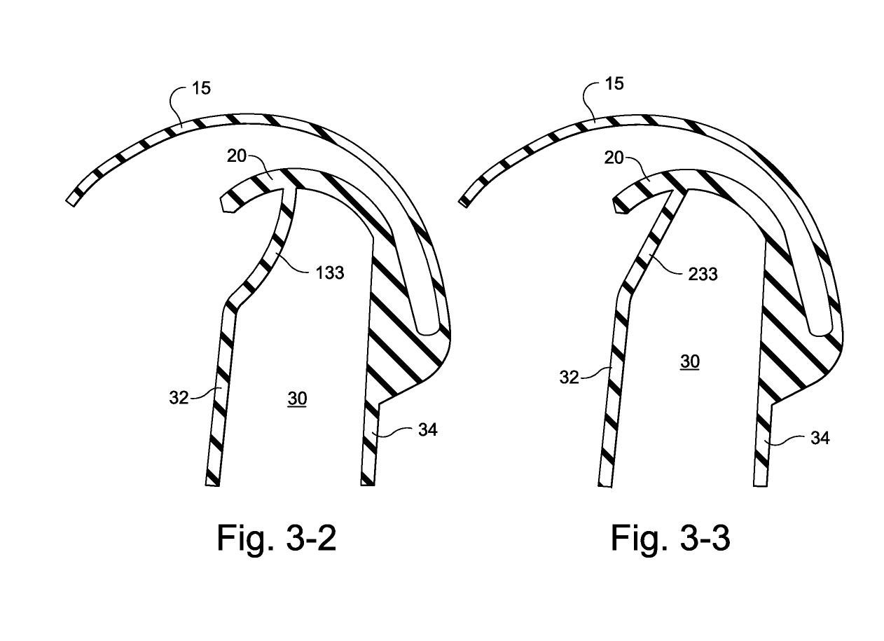

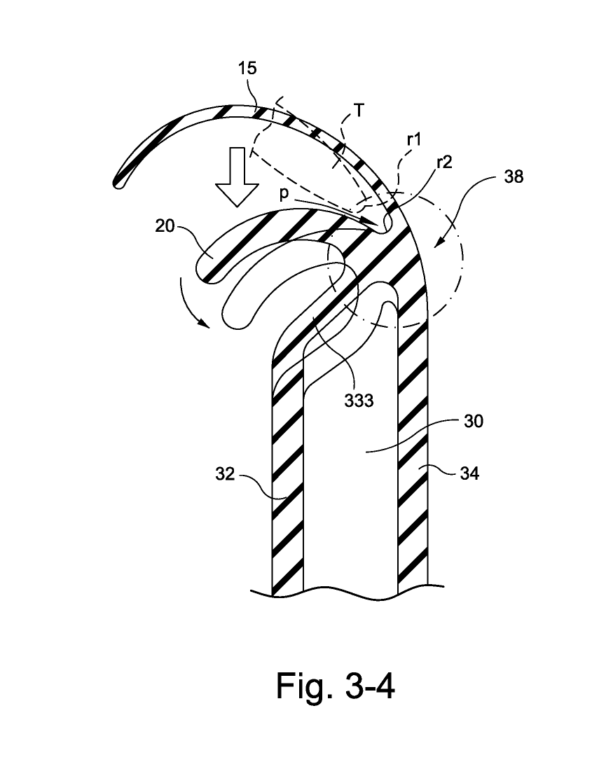

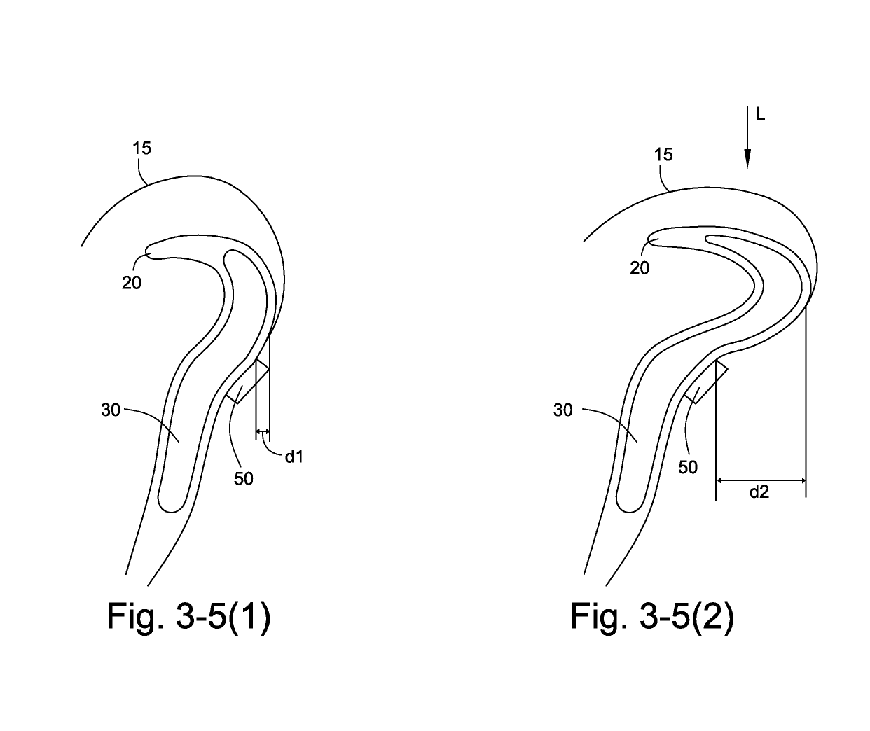

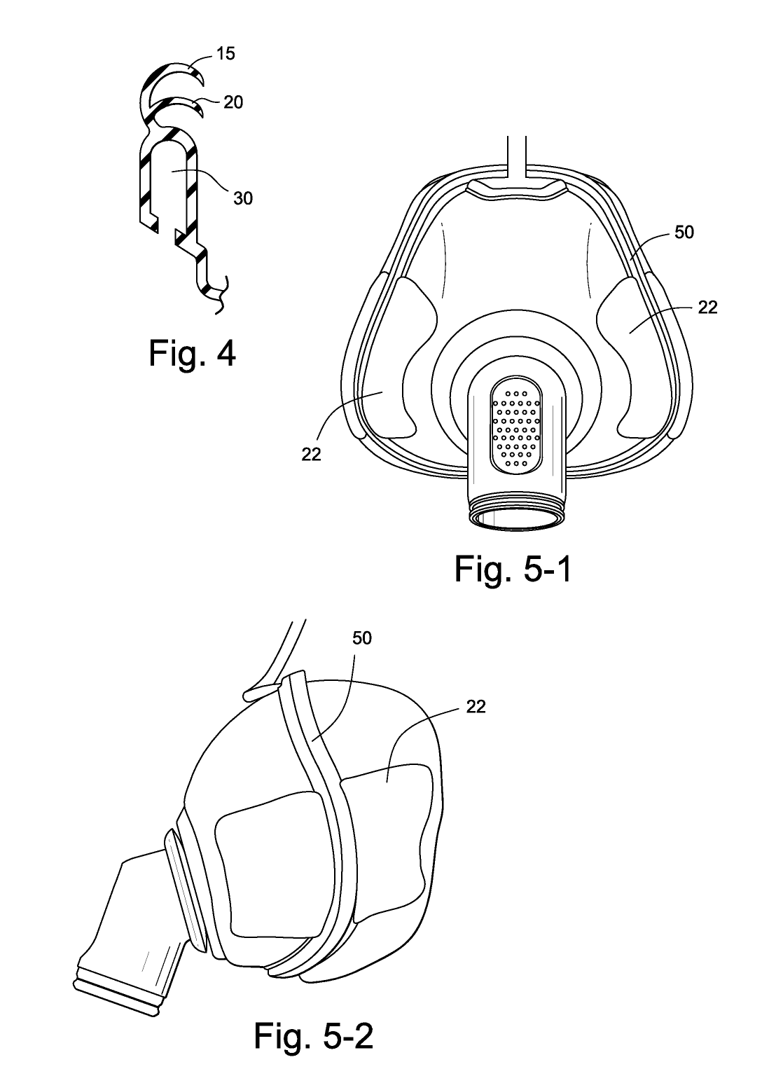

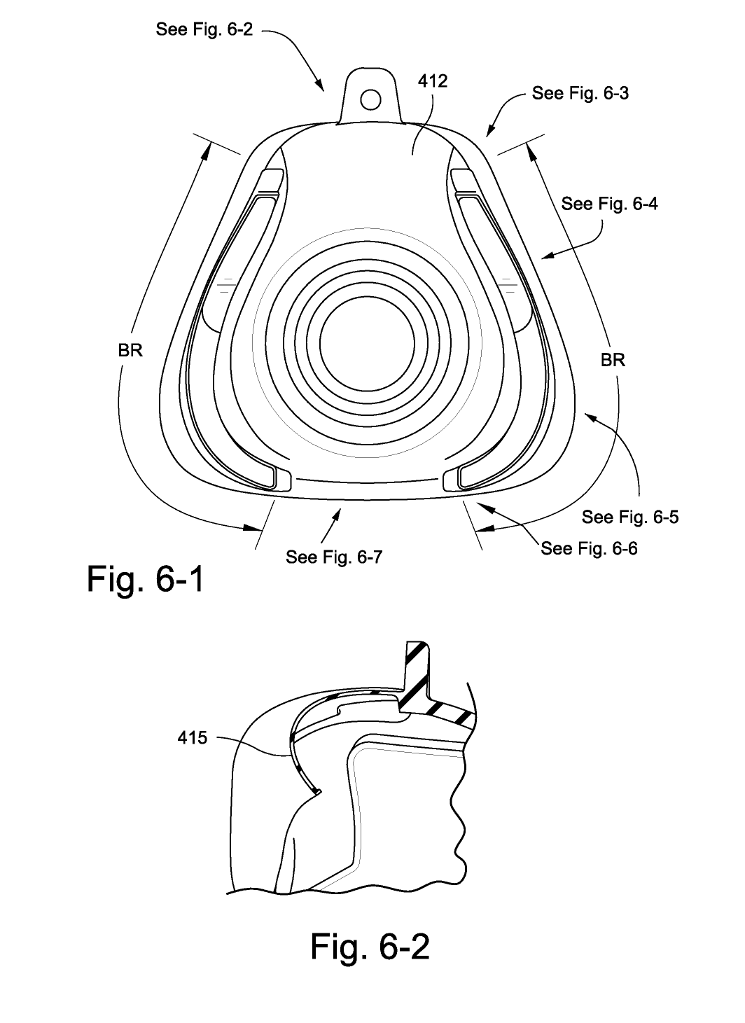

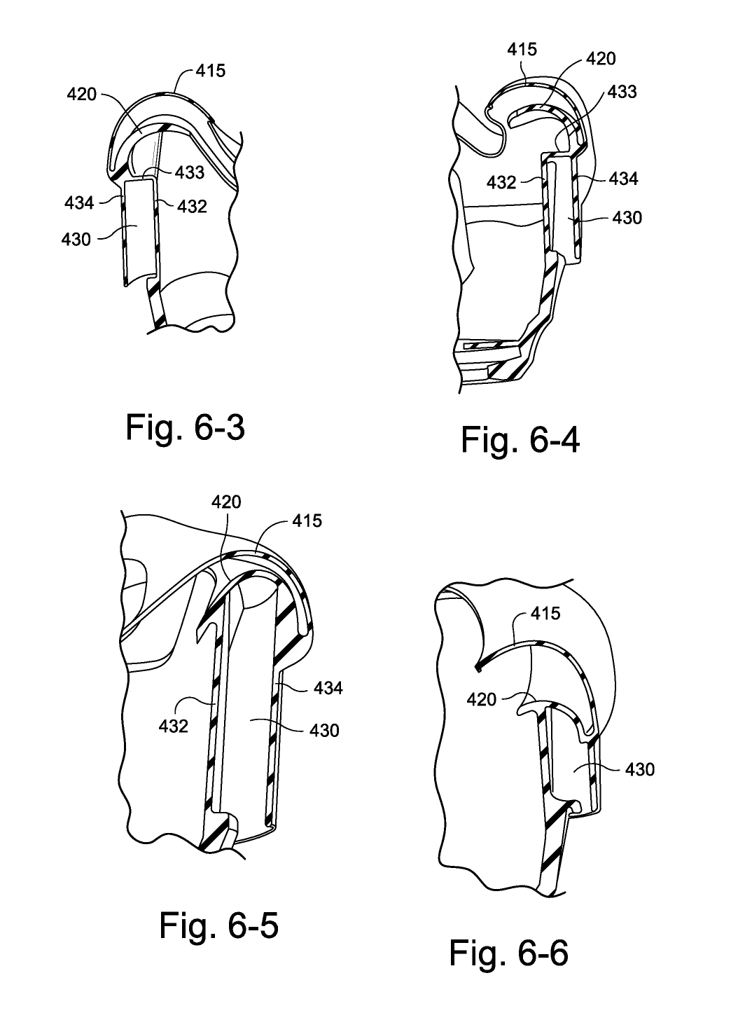

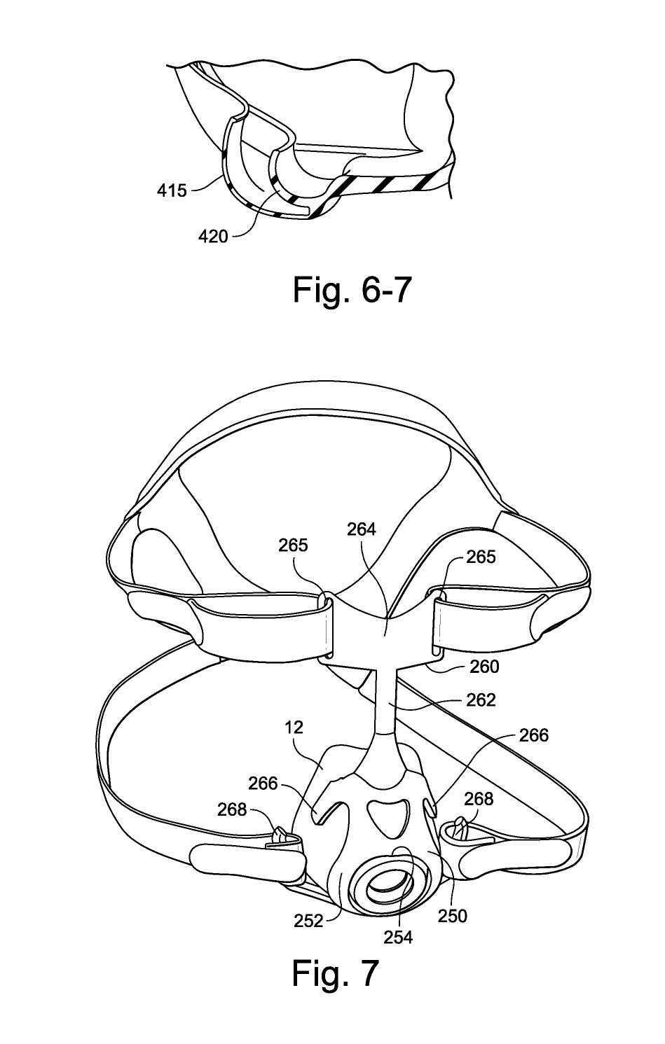

FIGS. 6-1 to 6-7 show a cushion 412 according to another example of the disclosed technology. As illustrated, the cushion 412 includes a single wall configuration, i.e., membrane 415 only, in the nasal bridge region (e.g., see FIG. 6-2), and the cushion includes a dual-wall configuration, i.e., membrane 415 and undercushion 420, in the remaining regions of the cushion, i.e., side of nose, upper and lower cheek, corner of nose, and upper lip regions (e.g., see FIGS. 6-3 to 6-7). In addition, a gel or super soft solid bladder 430 is provided in the side of nose, upper cheek, lower cheek, and corner of nose regions of the cushion (e.g., see FIGS. 6-1 and 6-3 to 6-6), e.g., see bladder region BR of cushion 412 in FIG. 6-1.

In contrast to the cushion 12 described above, the cushion 412 provides the bladder 430 further along the sides of the cushion, i.e., from the side of the nose region to the corner of nose region (see BR of cushion 412 in FIG. 6-1 as compared to the BR of cushion 12 in FIG. 1-2). The profile or configuration of the bladder may vary in different regions of the cushion. For example, the bladder in the side of nose region (e.g., see FIG. 6-3) includes a generally square profile, i.e., inner and outer side walls 432, 434 and upper wall 433 of bladder intersect at general right angles to one another. The bladder in the upper cheek region (e.g., see FIG. 6-4) includes a generally flat or planar profile, i.e., inner and outer side walls 432, 434 and upper wall 433 of bladder intersect such that upper wall 433 extends substantially perpendicular to support the undercushion 420. The bladder in the upper cheek region may transition to the undercushion at the nasal bridge region. The bladder in the lower cheek region (e.g., see FIG. 6-5) includes relatively straight side walls 432, 434 to facilitate molding, e.g., bladder walls are in the line of draw of the removable cores used to mold the cushion.

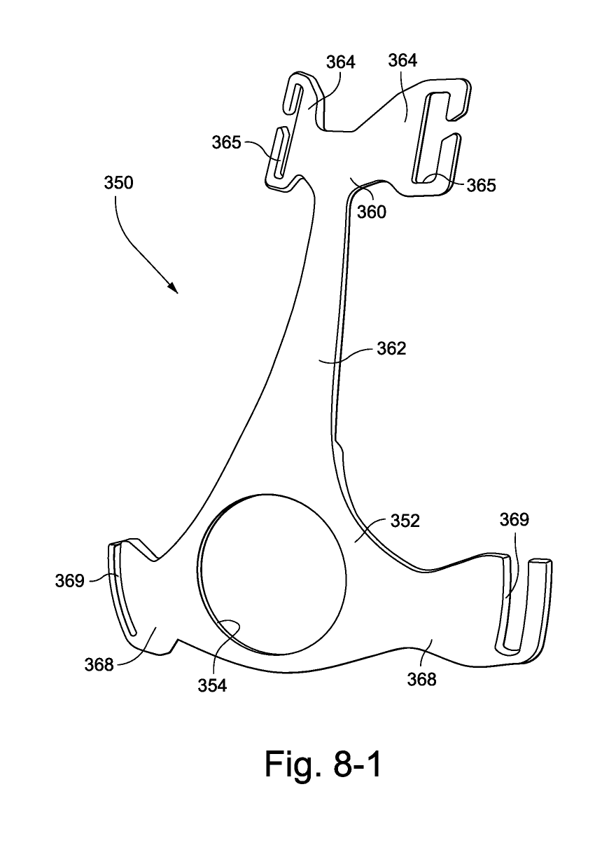

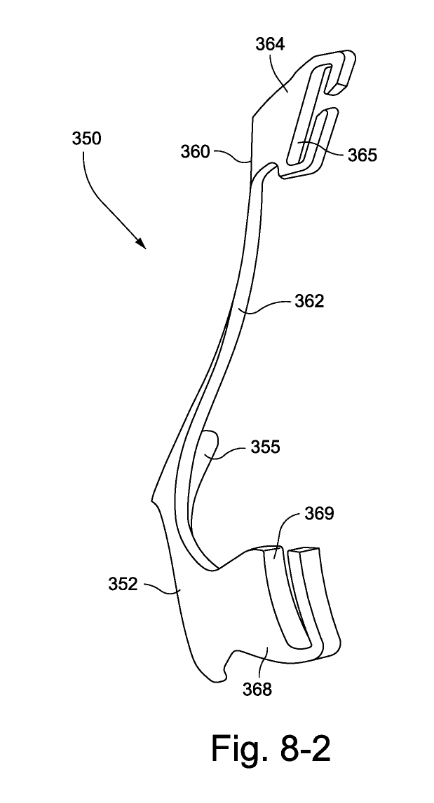

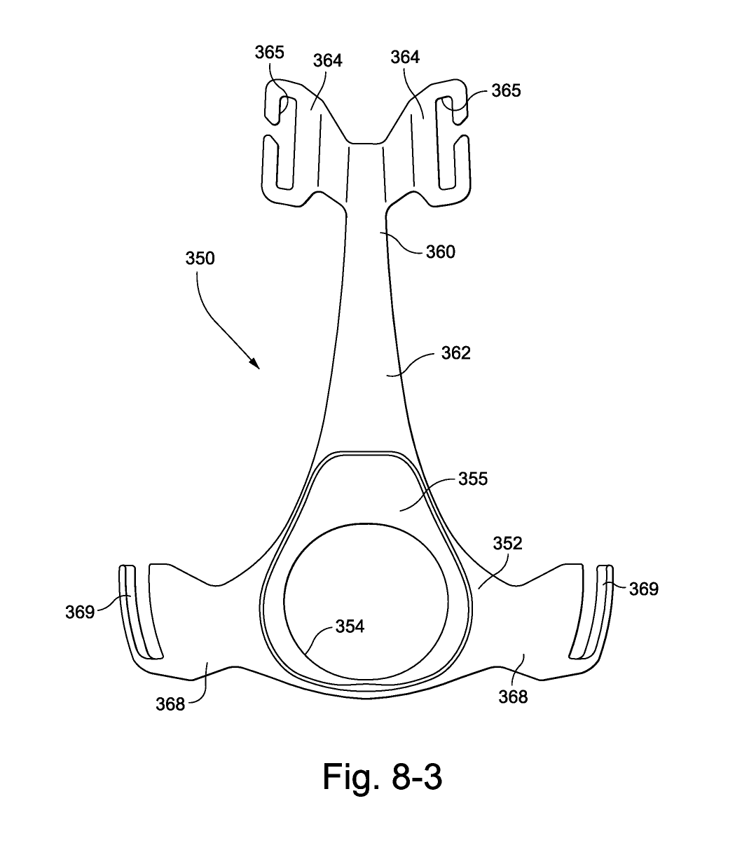

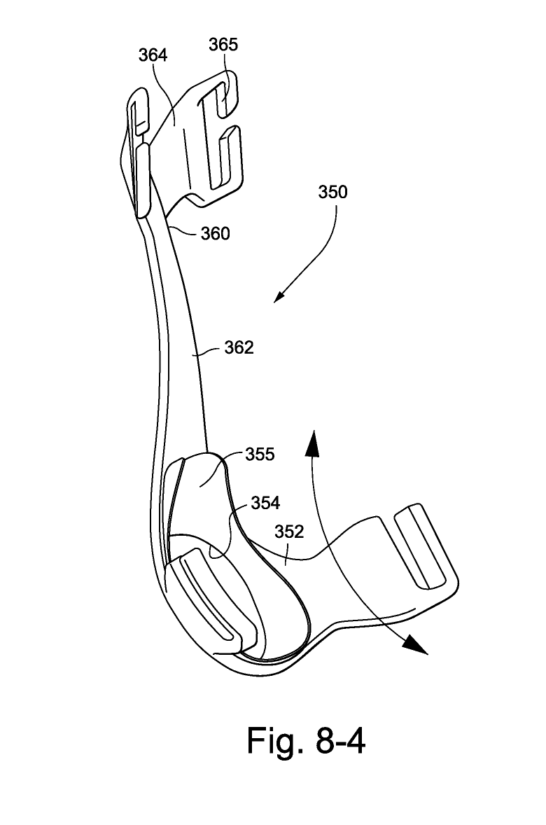

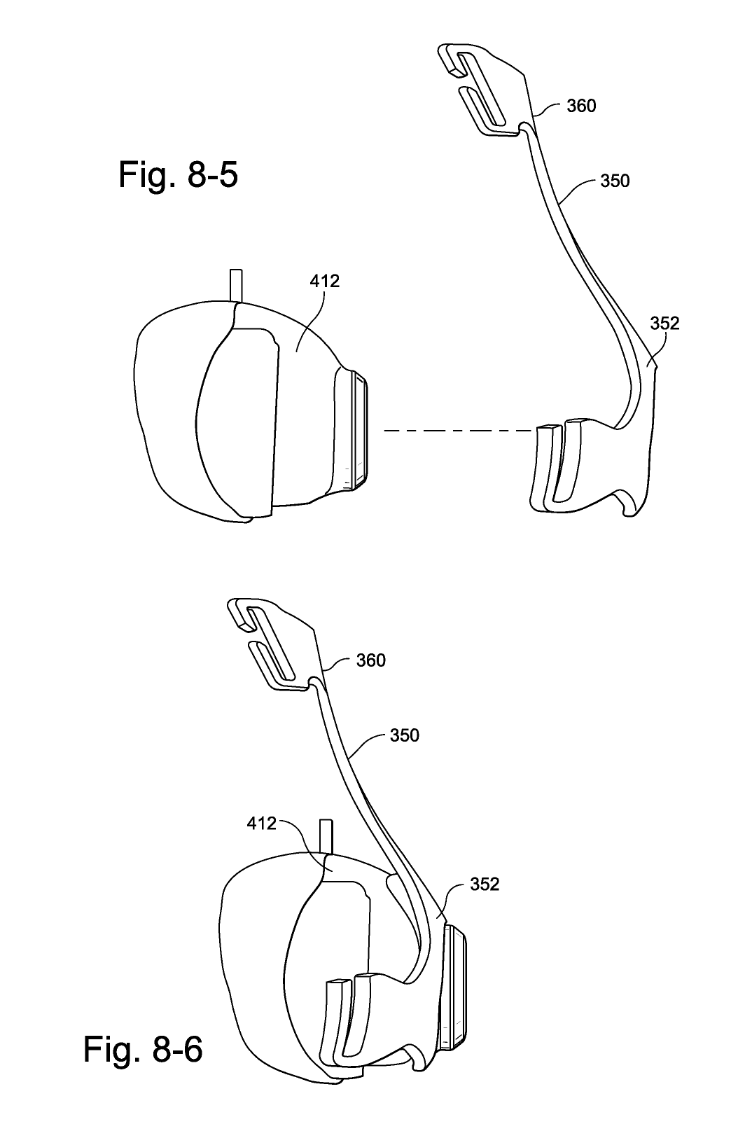

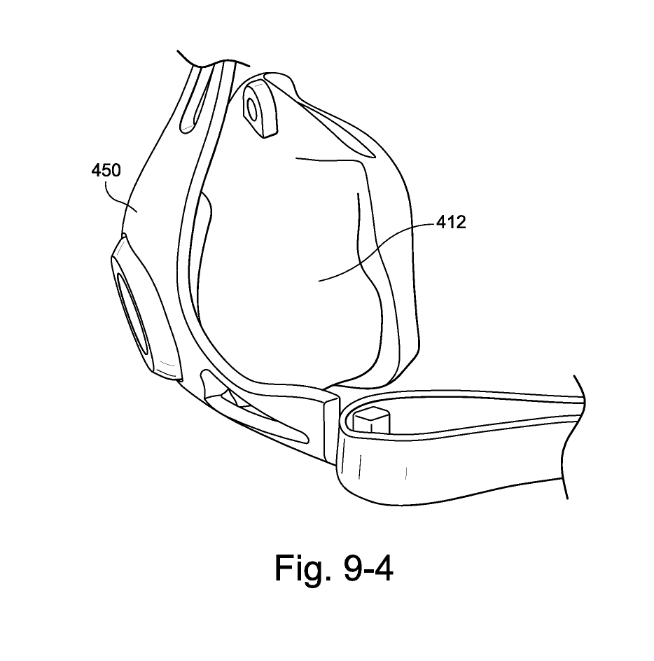

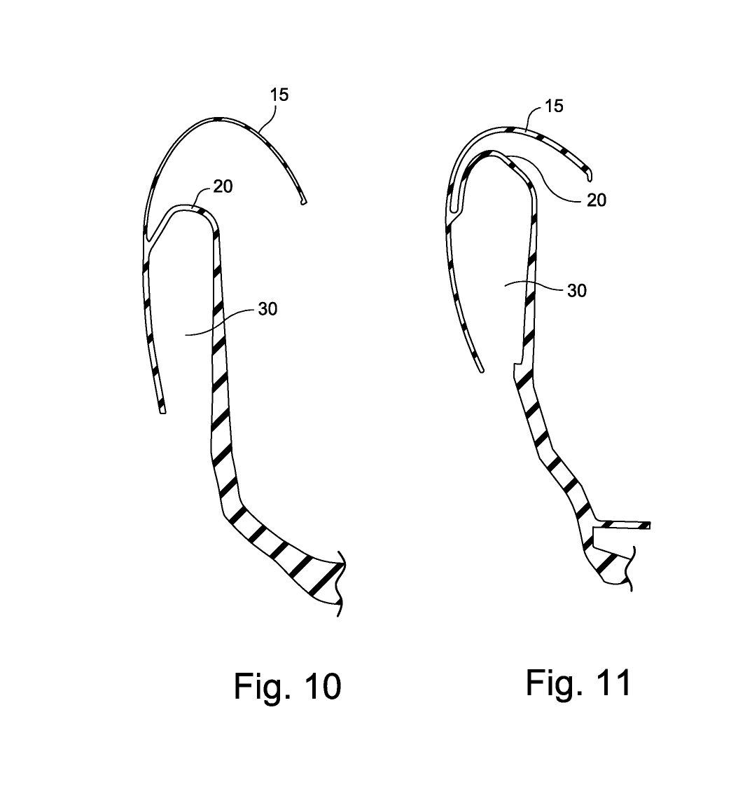

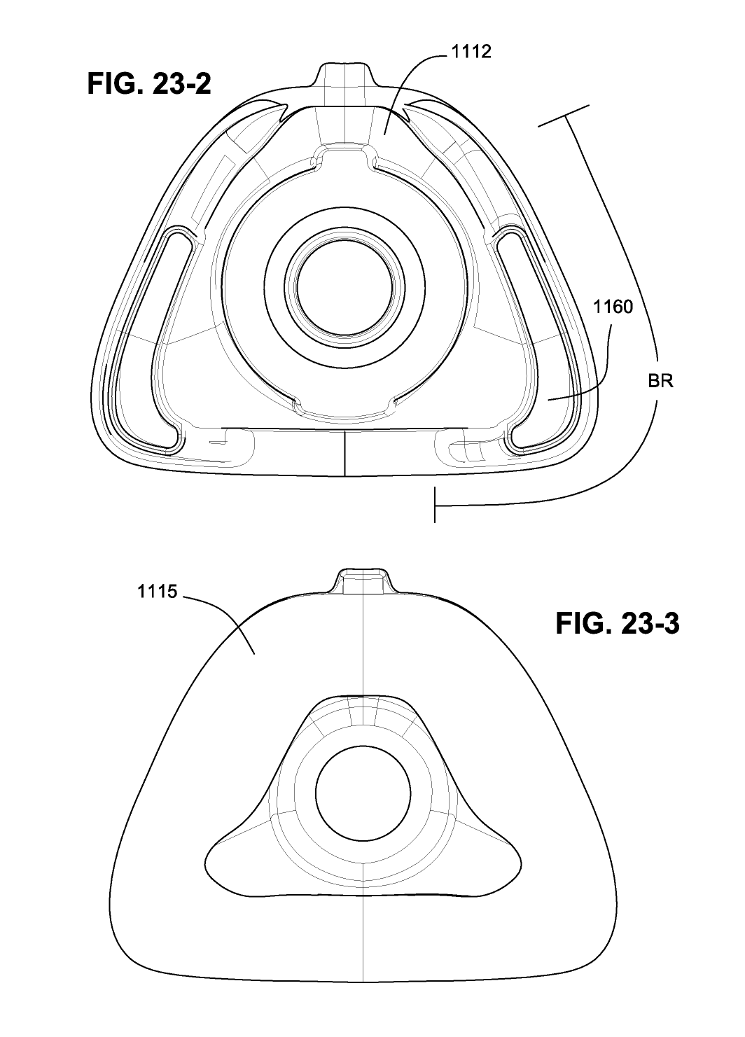

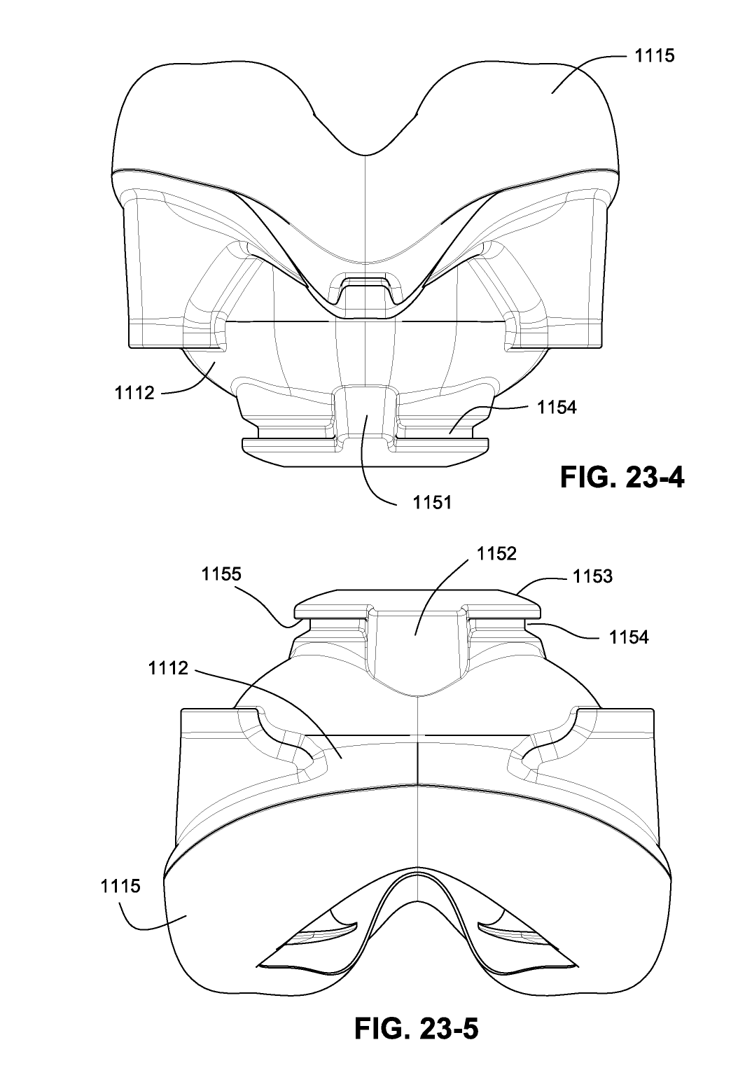

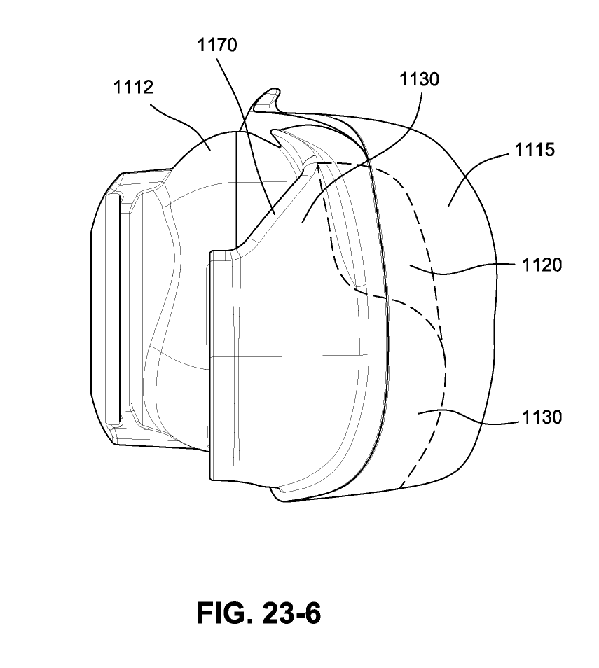

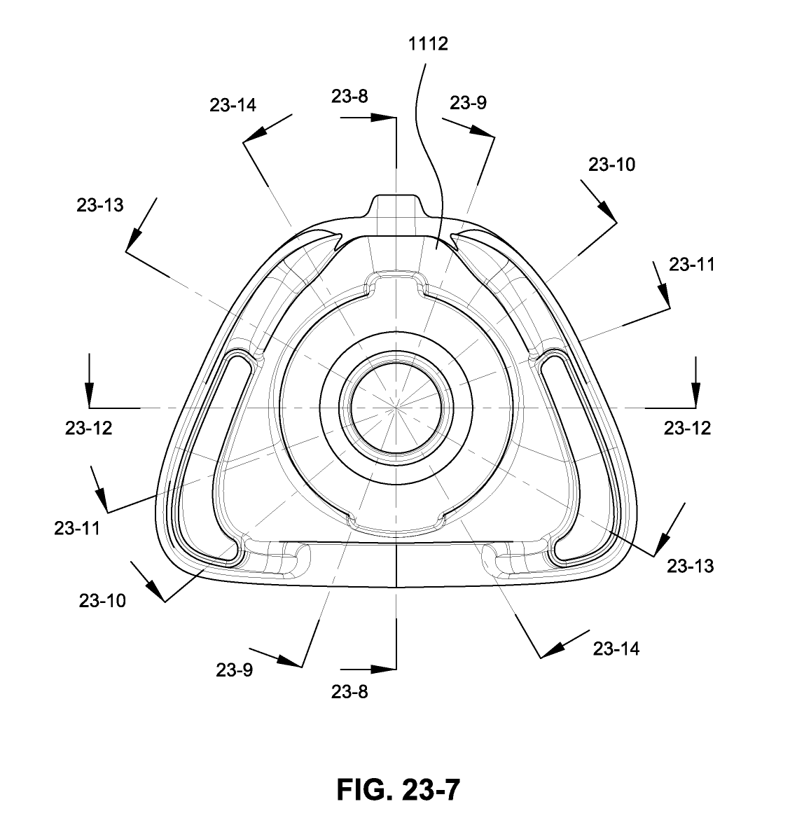

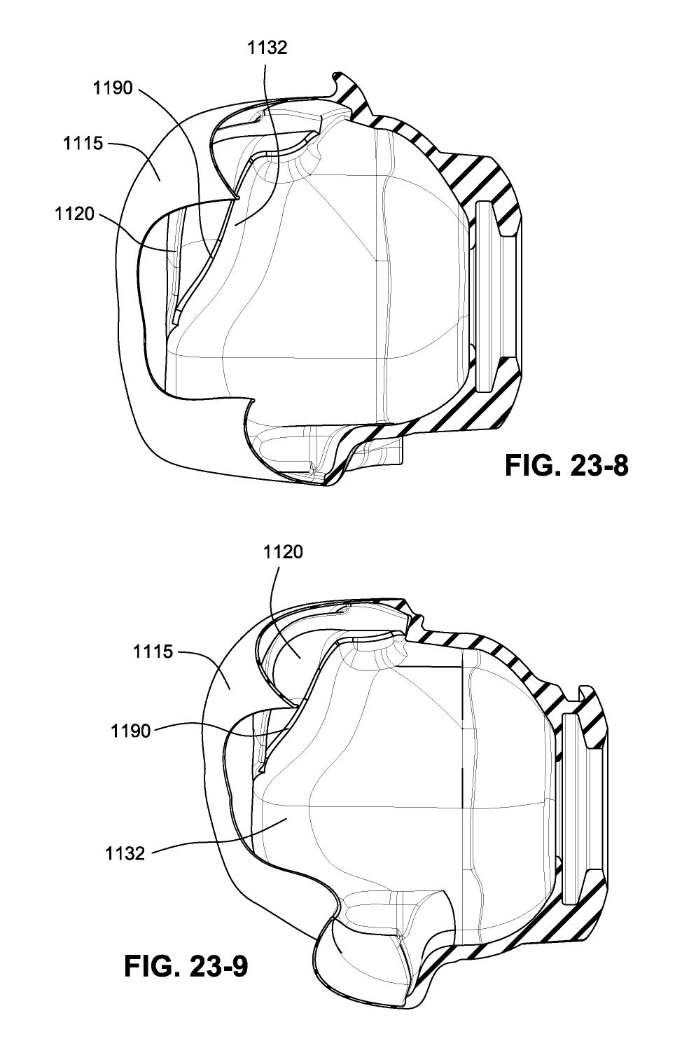

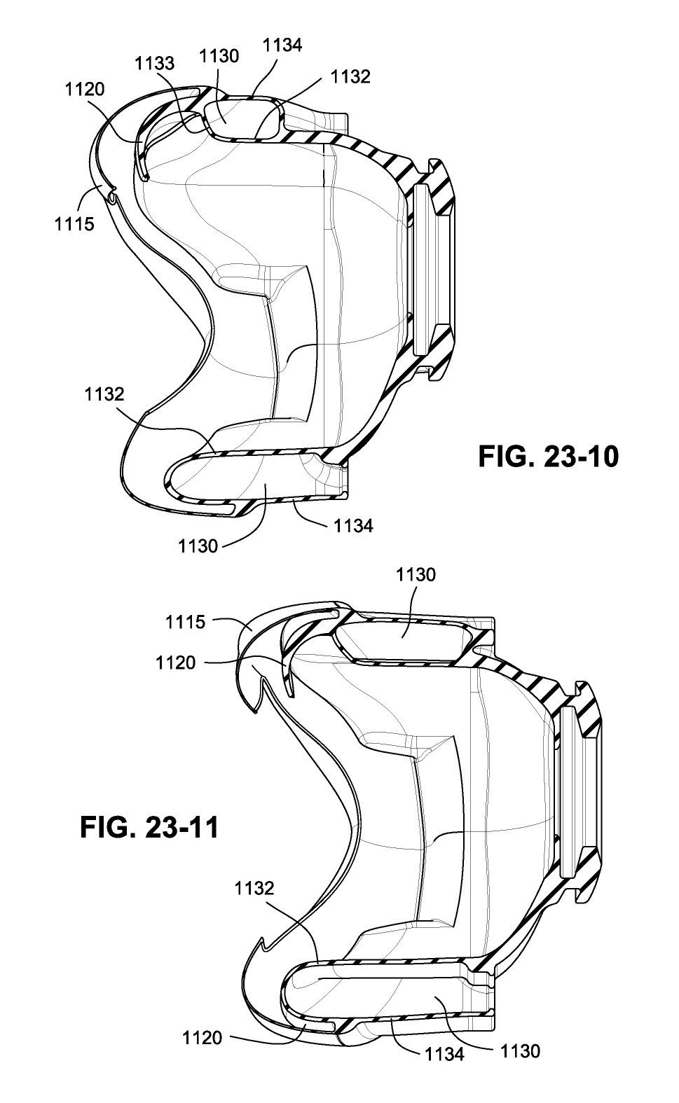

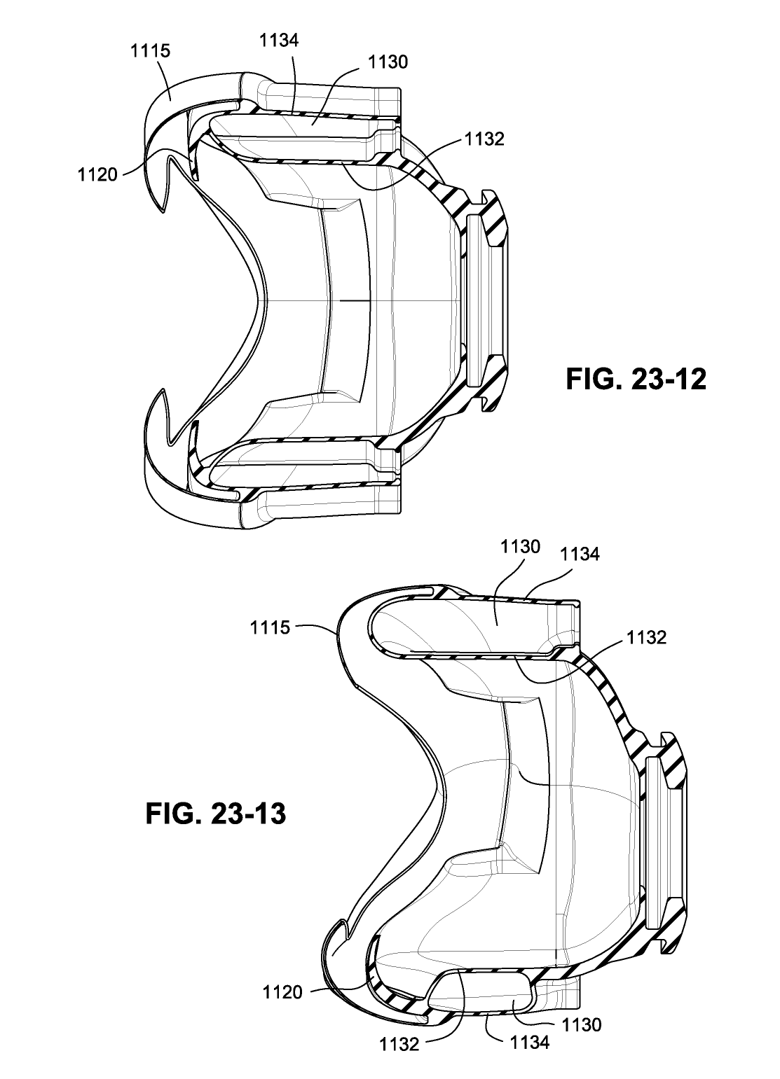

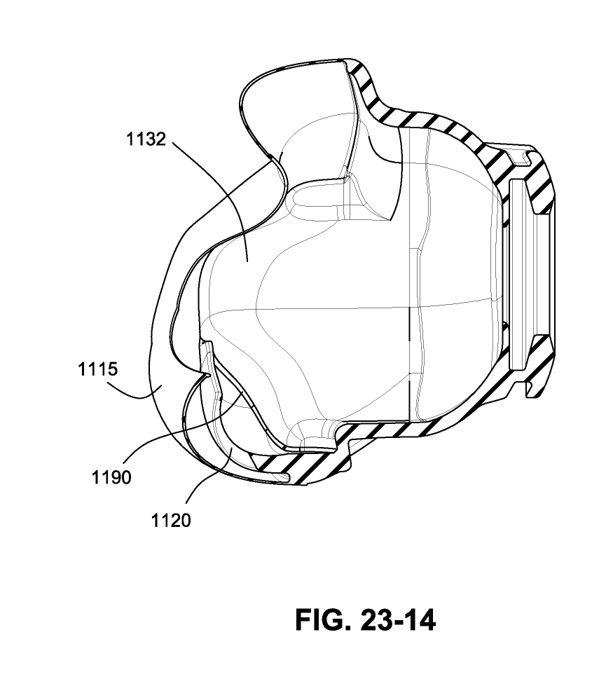

FIGS. 23-1 to 23-14 show a cushion 1112 according to another example of the disclosed technology. As illustrated, the cushion 1112 includes a single wall configuration, i.e., membrane 1115 only, in the nasal bridge region (e.g., see FIGS. 23-8 and 23-9), and the cushion includes a dual-wall configuration, i.e., membrane 1115 and undercushion 1120, in the remaining regions of the cushion, i.e., side of nose, upper and lower cheek, corner of nose, and upper lip regions (e.g., see FIGS. 23-10 to 23-14). In addition, a gel or super soft solid bladder 1130 is provided in the side of nose, upper cheek, lower cheek, and corner of nose regions of the cushion (e.g., see FIGS. 23-10 to 23-14). In contrast to cushion 12 described above, the cushion 1112 provides the bladder 1130 further along the sides of the cushion, i.e., from the side of nose region to the corner of nose region (see BR of cushion 1112 in FIG. 23-2 as compared to the BR of cushion 12 in FIG. 1-2). The bladder 1130 includes inner and outer side walls 1132, 1134 and upper wall 1133. The bladder 1130, when positioned adjacent to the sides of the nose region, may increase comfort of the cushion around the patient's nasal bridge. This arrangement may also enhance anthropometric tit range so that the cushion can accommodate patients having both high nose bridges (the filler in bladder 1130 may displace or change shape to accommodate the higher nose bridge) and low nose bridges (the filler in bladder 1130 may maintain the cushion on the patient's low nose bridge). In further contrast to the cushion 12 described above, the cushion 1112 also varies the depth of the bladder 1130 along the sides of the cushion. As shown in FIGS. 23-1 and 23-6, the depth of the bladder 1130 increases as the bladder extends from the nose region toward the cheek region. In an example of the side of nose region shown in FIG. 23-10, the bladder 1130 may have a depth of about 5-20 mm, preferably about 12-18 mm. In an example of the corner of nose region shown in FIG. 23-13, the bladder 1130 may have a depth of about 25-45 mm, preferably about 30-40 mm. The deeper pocket in the corner of nose region, as shown in FIGS. 23-13 and 23-14, may improve the seal of the cushion in the corners of the nose, provide a tactile region for the patient to touch, and may also improve the aesthetic of the cushion. Furthermore, the deeper pocket may act as a visual indicator for alignment, i.e. larger gel portions in the corners of the nose to indicate the cushion is to be positioned a certain way. The changing depth of the bladder 1130 can be seen at wall 1170, as shown in FIG. 23-6. The wall 1170 may be sloped or angled in order to reduce the depth of gel near the sides of the patient's nose thereby reducing the compressive seal near the sides and bridge of the patient's nose, while increasing the depth of the gel near the corners of the patient's nose. In addition, the patient contacting face of the bladder slopes away from direct interaction with the patient at the sides of the nose, as can be seen at the intersection line 1190 in FIG. 23.8. This allows room for the undercushion which eventually replaces the intersection 1190, as shown in the upper section of FIGS. 23-10 and 23-14. Such an arrangement allows the seal at the sides of the nose to be a rolling or tensile type seal rather than a compression type seal. Furthermore, the wall 1170 may be sloped or angled to facilitate demolding of the bladder.

As shown in FIGS. 23-1, 23-4 and 23-8, a frame connecting member 1150 of the cushion may comprise a first tab 1151, a second tab 1152, and a channel 1154. The frame connecting member 1150 may be adapted to receive a portion of a frame, thereby connecting the cushion to the frame in order to position and stabilize the cushion on the patient's face. The frame may comprise a tongue, flange or other structure adapted to be received by the channel 1154. The frame connecting member 1150 may further include a front face 1153 having a surface that curves or slopes radially outwardly towards the outer edges of the frame connecting member 1150 to allow for easy connection of the frame. The channel 1154 may be bound by a wall 1155 on the frame connecting member 1150. The wall 1155 may be substantially horizontal or perpendicular to the direction that the frame is connected to the frame connecting member 1150. This arrangement may prevent or increase the difficulty of disassembling the frame from the frame connecting member 1150. The first tab 1151 and the second tab 1152 may aid in aligning the frame with the frame connecting member 1150, and may also prevent the frame from rotating relative to the cushion via channel 1154.

As shown in FIGS. 23-1 and 23-2, a bladder opening 1160 may be formed as a slot or hole adapted to receive a gel or super soft solid. The bladder 1130 may be formed by placing a portion of a tool (not shown) into a mold, and when the molding process is complete, the tool may be removed through the bladder opening 1160. The width of the bladder opening 1160 may be about 2-10 mm, preferably about 4-7 mm. A bladder opening width on the higher side of this range may be desirable, since it is easier to remove the tool from the mold without damaging the bladder when the width of the bladder opening is larger.

FIG. 23-10 shows a cross section through cushion 1112 at the side of nose and corner of nose regions. In the side of nose region (shown in the upper section of FIG. 23-10), the bladder 1130 is more distant from the membrane 1115 (as compared to the corner of nose region in the lower section of FIG. 23-10) and is essentially replaced by the undercushion 1120. In this region, less support from the gel or super soft solid in the bladder may be required and instead support is provided by the undercushion 1120 (or in an alternative example, air pressure) to form a tension and/or rolling type seal. In the corner of nose region, compression seals are better tolerated than elsewhere and are useful for mask stability. Consequently, the gel or super soft solid in the bladder may function to form a compression type seal with the patient's face. This is shown in the lower section of FIG. 23-10 where the bladder 1130 extends without presence of the undercushion 1120.

The location where the membrane connects with the bladder or undercushion forms a relatively stiff region due to the thickness of the material. It is therefore preferable to keep this area away from the patient's face as it may cause localized pressure points during use.

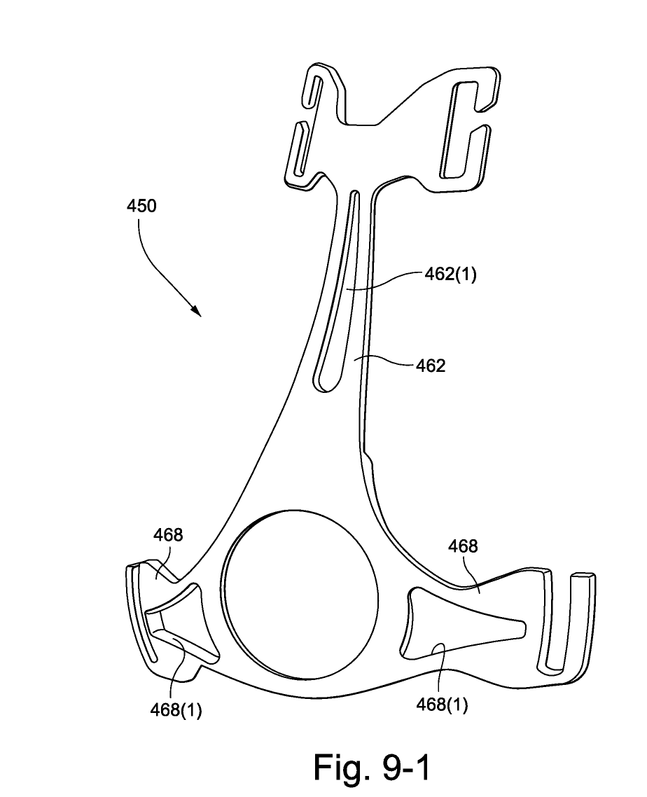

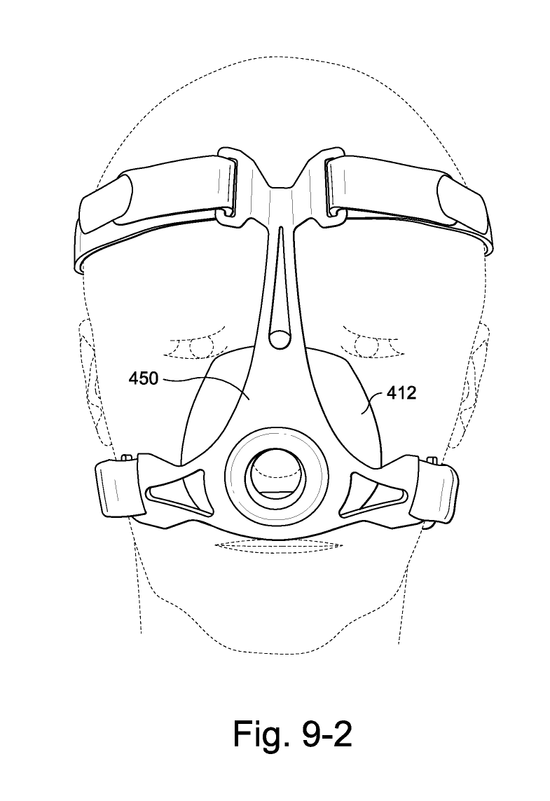

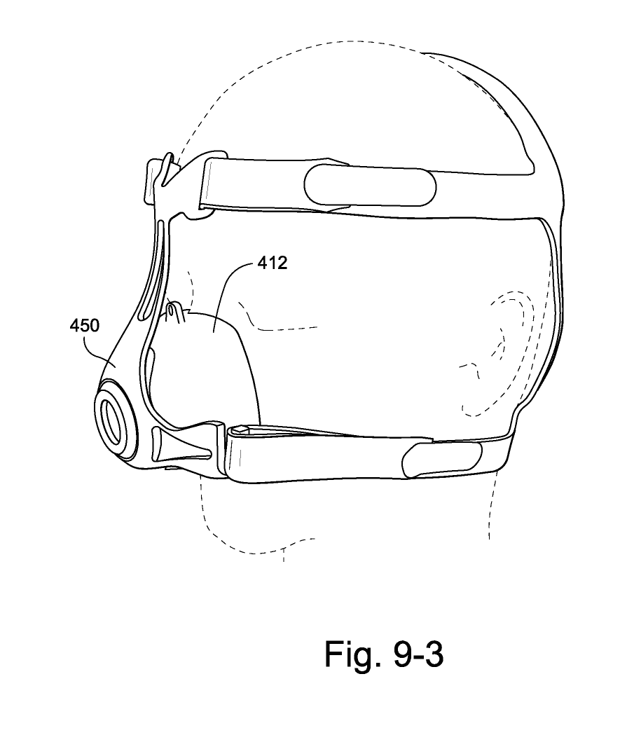

FIGS. 24-1 to 24-14 show a cushion 1212 according to another example of the disclosed technology. As illustrated, the cushion 1212 includes a single wall configuration, i.e., membrane 1215 only, in the nasal bridge region (e.g., see FIGS. 24-8 and 24-9), and the cushion includes a dual-wall configuration, i.e., membrane 1215 and undercushion 1220, in the remaining regions of the cushion, i.e., side of nose, upper and lower cheek, corner of nose, and upper lip regions (e.g., see FIGS. 24-9 to 24-14). In addition, a gel or super soft solid bladder 1230 is provided in the side of nose, upper cheek, lower cheek, and corner of nose regions of the cushion (e.g., see FIGS. 24-10 to 24-13). The bladder 1230 includes inner and outer walls 1232, 1234 and upper wall 1233.

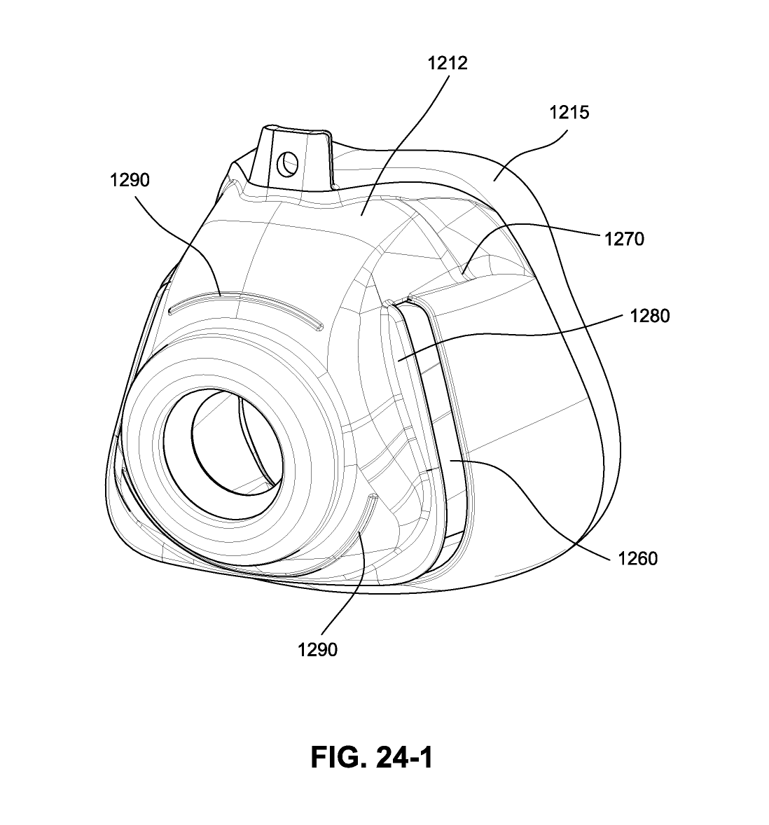

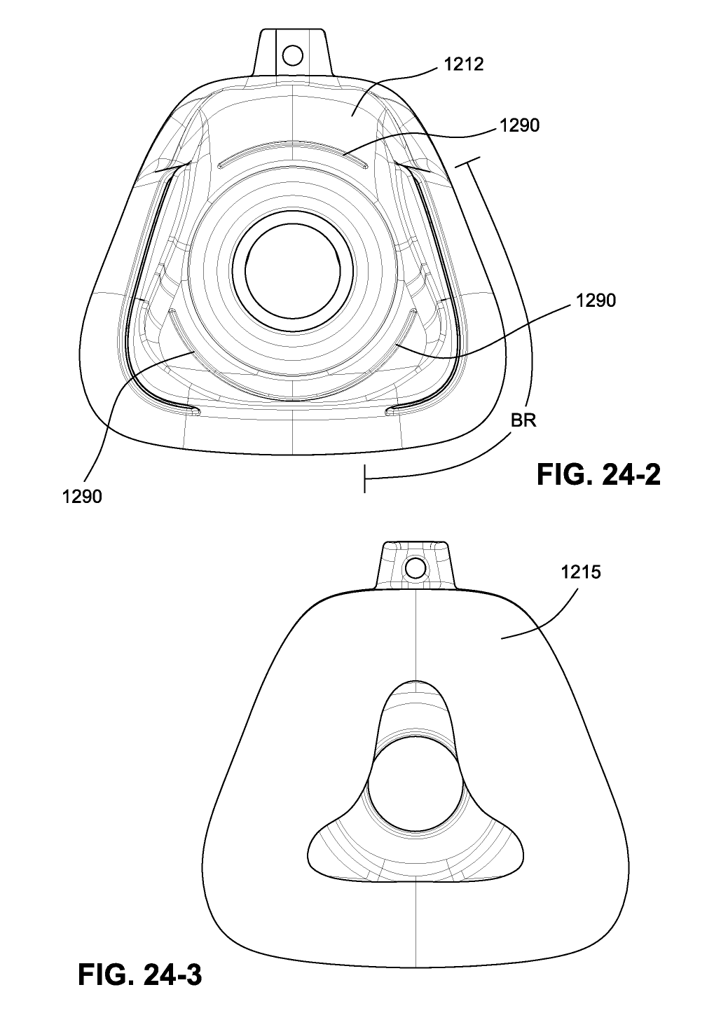

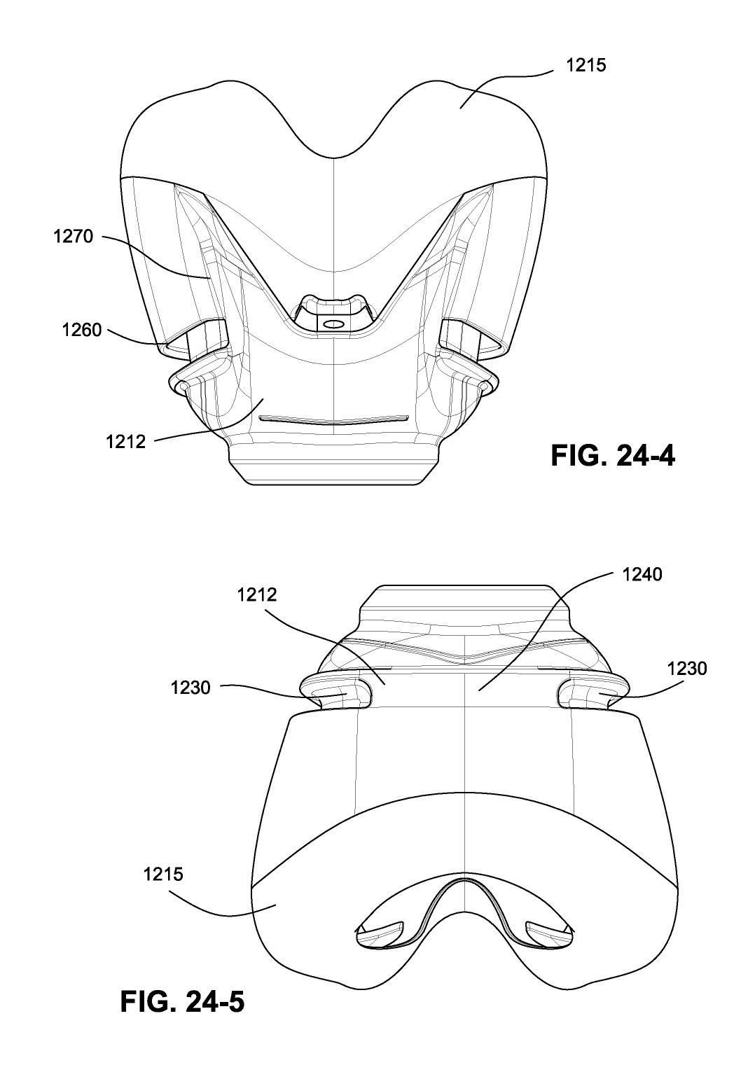

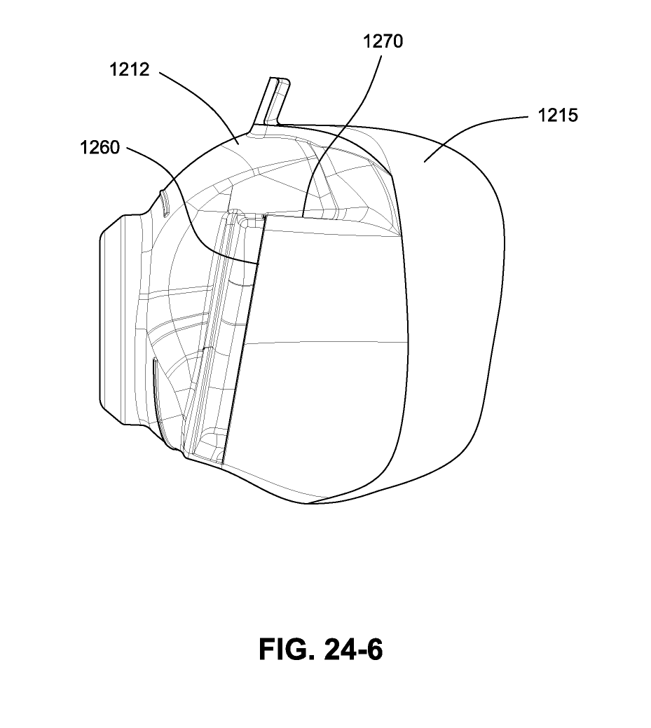

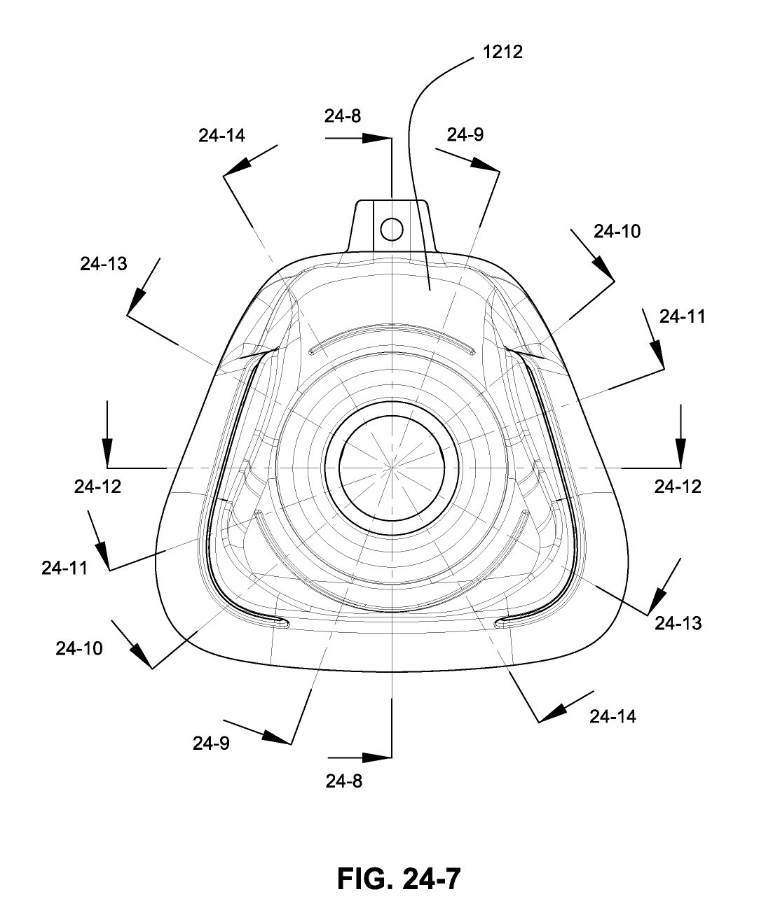

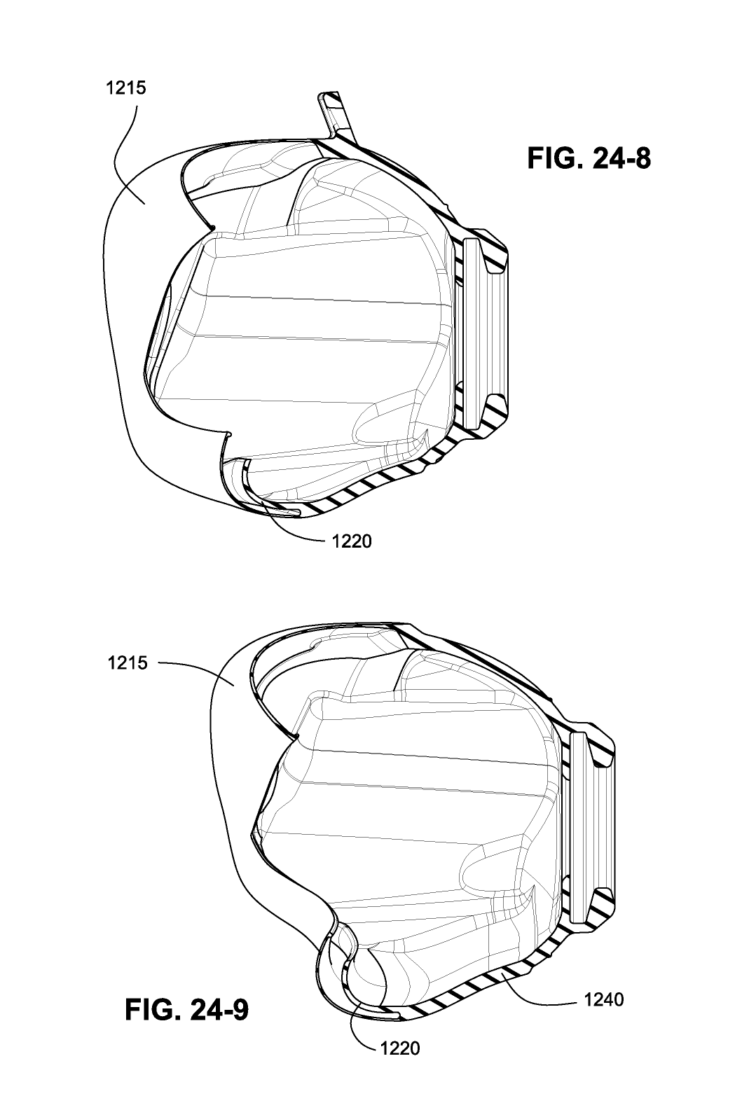

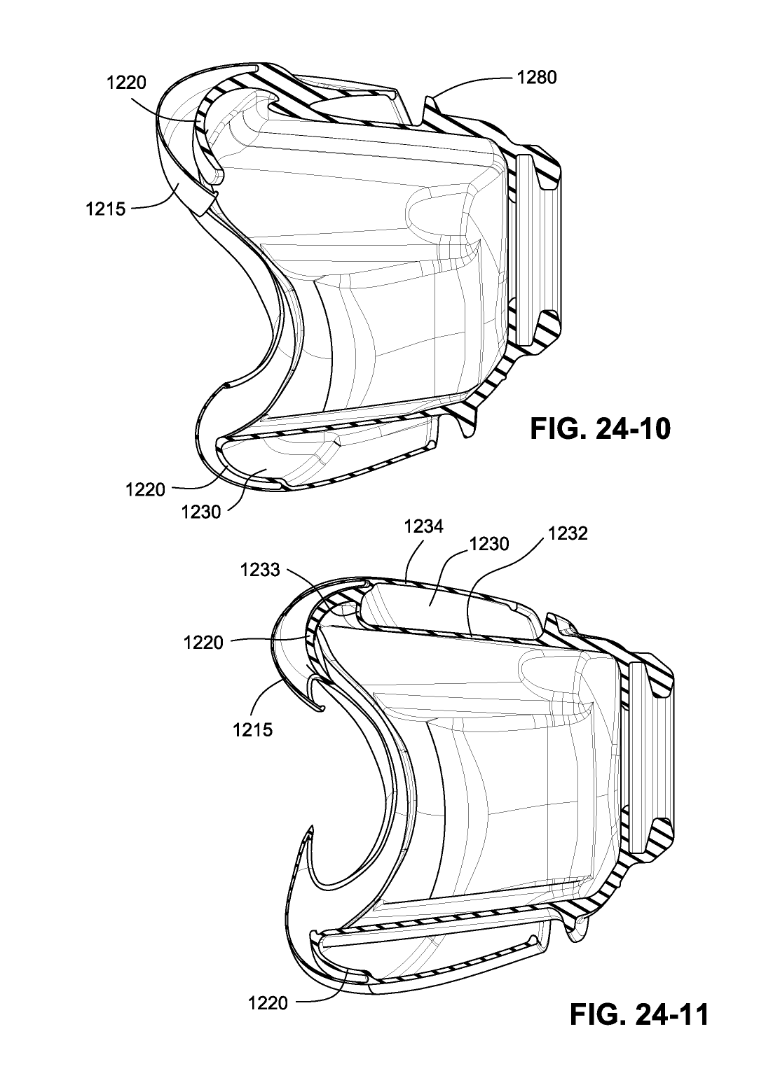

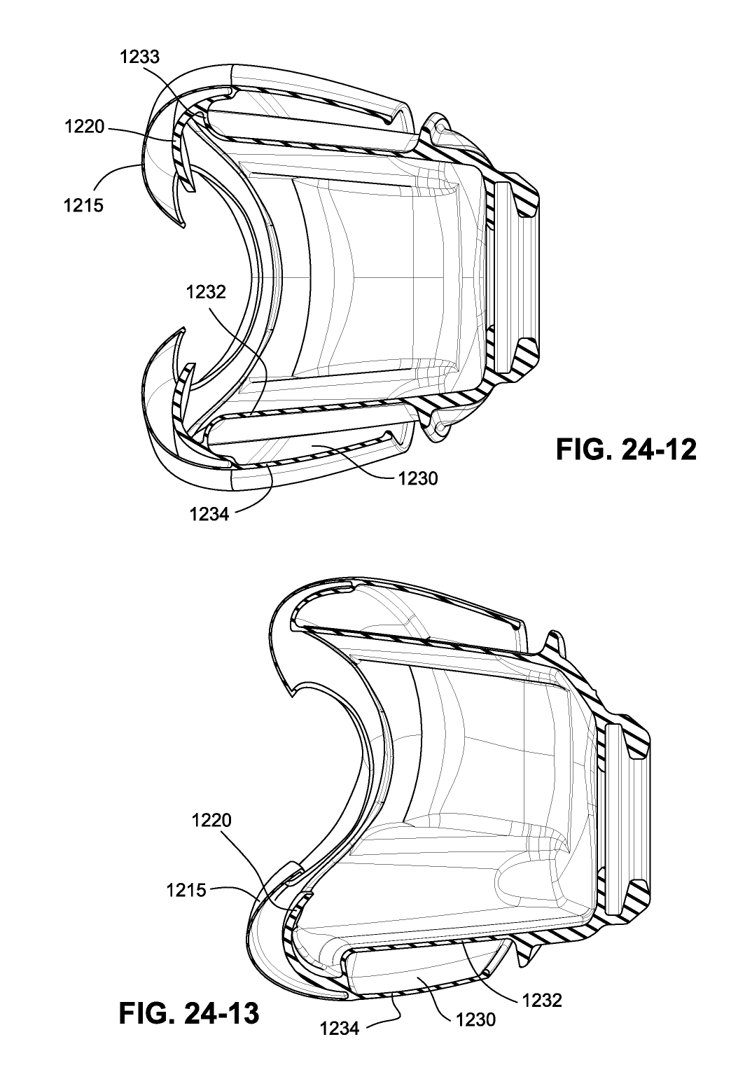

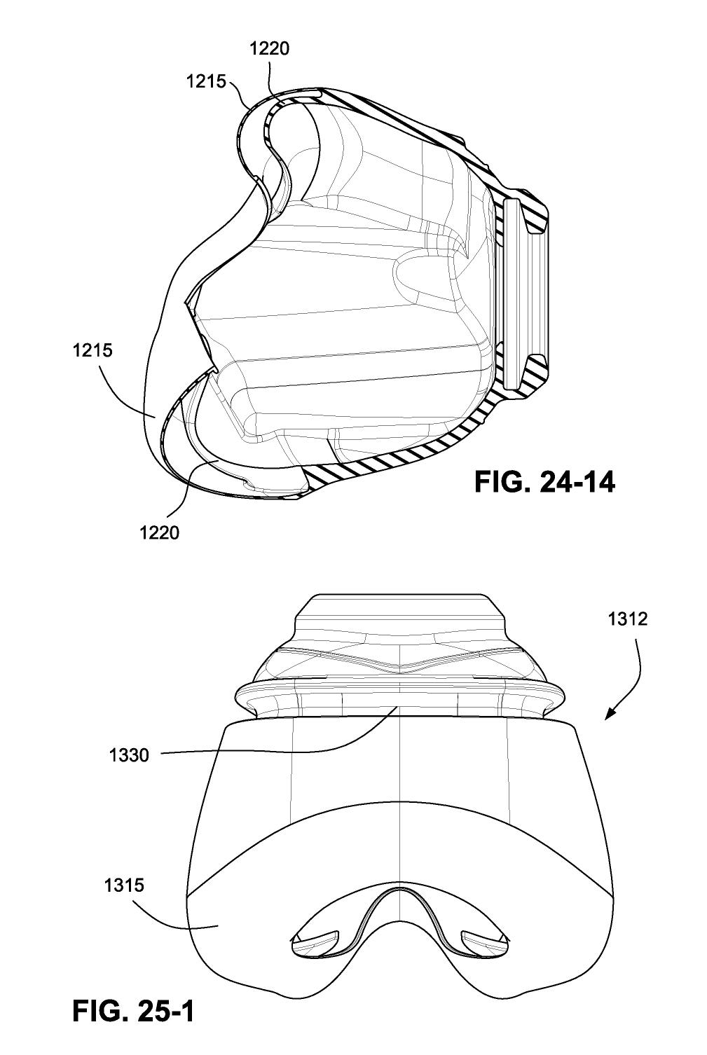

In contrast to the cushion 12 described above, the cushion 1212 provides the bladder 1230 further along the sides of the cushion, i.e., from the upper cheek region to the corner of nose region (see BR of cushion 1212 in FIG. 24-2 as compared to the BR of cushion 12 in FIG. 1-2). Similar to the cushions 12, 412 and in contrast to the cushion 1112, the opening of the bladder 1230 may remain constant along the sides of the cushion relative to the front or the non-patient contacting side in the illustrated example—for example, wall 1270 of cushion 1212 may be substantially perpendicular to the bladder opening 1260. However, the depth of the bladder 1230 may vary along the sides of the cushion; therefore, the profile of the bladder on the patient contacting side is variable. The bottom of the cushion extends continuously into dividing wall 1240 which is situated between the bladders 1230, as seen in FIGS. 24-5 and 24-9, to facilitate molding and improve the aesthetic of the cushion by creating a smooth continuous surface between the bladder openings.

As best shown in FIGS. 24-1, 24-2 and 24-4, the cushion 1212 may further comprise a ledge, flap or cap region 1280 adapted to provide an aesthetic cover over the bladder opening 1260. The ledge 1280 may extend over the entire width of the bladder opening 1260 or alternatively only over a portion of bladder opening 1260. The ledge 1280 may be adapted to engage with a flange on a frame in order to position the flange between the ledge 1280 and the bladder opening 1260, thereby sealing the bladder opening 1260 and engaging the cushion with the frame.

As best shown in FIGS. 24-1 and 24-2, the cushion 1212 may comprise a bump or lip 1290 which may provide an indication for alignment of the cushion. The bump or lip 1290 may also provide for engagement with a frame, a vent or an elbow. For example, the lip 1290 may be positioned to interface with grooves on a frame. The lip 1290 may be positioned vertically above an air inlet of the cushion. Additionally, as shown, there may be numerous lips 1290 positioned around the air inlet of the cushion.

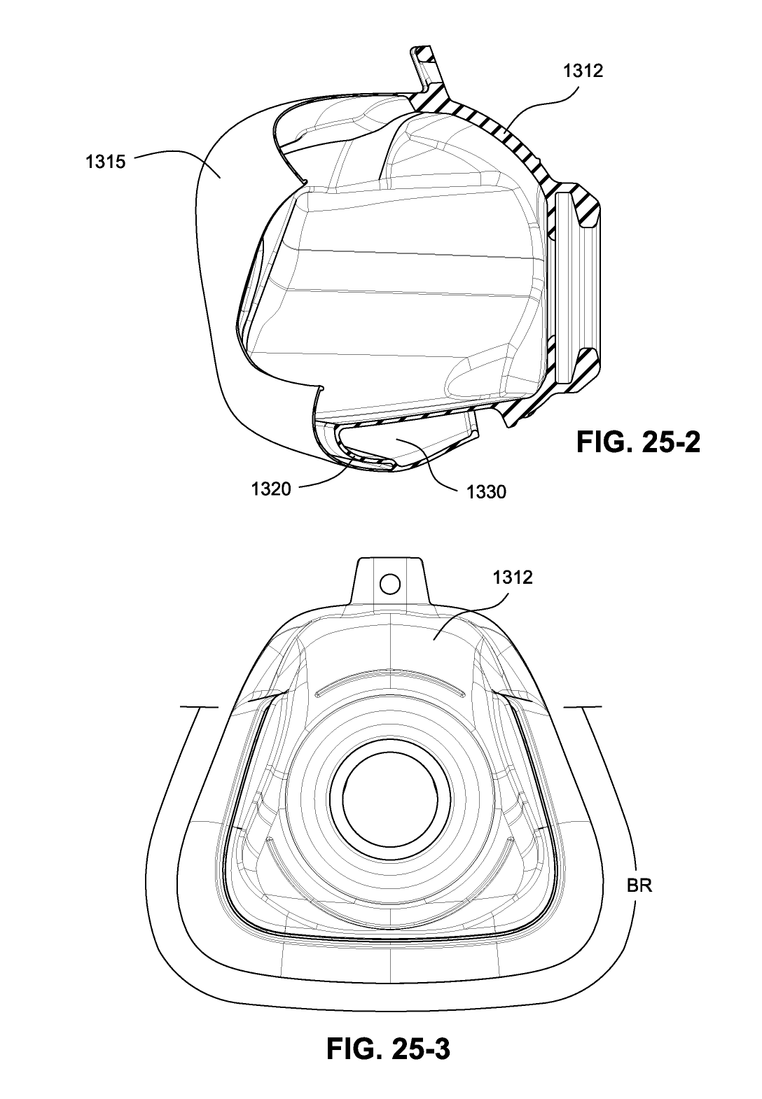

FIGS. 25-1 to 25-3 show a cushion 1312 according to another example of the disclosed technology. Cushion 1312 is substantially similar to cushion 1212 of FIGS. 24-1 to 24-14. The difference between cushion 1212 and cushion 1312 is a gel or super soft solid bladder 1330 is provided in the side of nose, upper cheek, lower cheek, corner of nose, and upper lip regions of the cushion (see BR of cushion 1312 in FIG. 25-3 as compared to the BR of cushion 1212 in FIG. 24-2). In contrast to the cushion 1212 described above, the bladder 1330 extends continuously across the upper lip region, as shown in FIGS. 25-1 to 25-3. The arrangement of a continuous bladder 1330 may be preferable to multiple separate bladders as it may be easier and more cost effective to mold and fill the bladders. Furthermore, comfort may be enhanced and/or the cushion may fit a broader anthropometric range when the conformable material of the bladder is included in the upper lip region.

### 2.1 Frame Connection

The non-face-contacting or frame side of the cushion includes one or more interfacing structures adapted to interface or otherwise removably connect to a frame. In the illustrated example shown in FIGS. 1-1 to 1-5, the cushion includes elongated protrusions 22 along the sides of the cushion adapted to engage or interlock with the frame. However, it should be appreciated that the cushion may be connected or interlocked with the frame in other suitable manners.

For example, FIGS. 5-1 and 5-2 show an example in which the cushion includes an elongated protrusion 22 on each side of the cushion, each protrusion including a slot to receive or interlock with the frame 50 so as to retain the cushion to the frame.

In an example, the protrusions along the sides of the cushion may be pockets of gel or super soft solid. In an example, the pocket of gel or super soft solid extending between the elbow connection and the frame may be for the patient to touch so as to engage or entice the patient. In an example, the pocket of gel or super soft solid between the frame and the sealing membrane may be adapted to support the sealing membrane.

### 2.2 Elbow Connection

The non-face-contacting side of the cushion includes an opening 25 adapted to receive or otherwise communicate with an elbow for delivering breathable gas. As best shown in FIGS. 1-6 and 1-7, an annular flange 26 defines the opening and is structured to engage or interlock with the elbow directly or with an optional swivel ring adapted to couple the elbow. Also, an interference lip seal 27 may be provided axially inwardly from the opening and is adapted to seal against an end of the elbow and/or the swivel ring.

In a further example, the elbow may be a flexible bellows arrangement.

Alternatively, there may be no elbow and the air delivery tube may connect directly to the sealing interface or frame.

### 2.3 Bladder

In the illustrated example, the undercushion 20 defines a portion of the gel or super soft solid bladder 30, i.e., bladder may be referred to as a gel or super soft solid fillable undercushion. As best shown in FIGS. 1-7, 1-8, 1-12, and 1-13, each gel or super soft solid bladder is defined by spaced apart inner and outer walls 32, 34 and the undercushion 20 that interconnects the inner and outer walls 32, 34. The membrane 15 and undercushion 20 extend from the outer wall 34, and the free end or edge of the undercushion 20 intersects the inner wall 32. As illustrated, the free end of the undercushion 20 overhangs the inner wall of the bladder which provides a lead-in feature or undercushion lip 20(1) to encourage rolling of buckling of the bladder wall in use. In an example, the undercushion intersects with the inner wall of the bladder, and the inner wall includes a curved or slanted portion adapted to provide a feature to control buckling of the inner wall in use. In an example, the free end of the undercushion 20 may not overhang the inner wall 32. In the illustrated example, the walls of the gel or super soft solid bladder have a generally constant cross-section along most of its length. However, it should be noted that the bladder need not have a constant cross section.

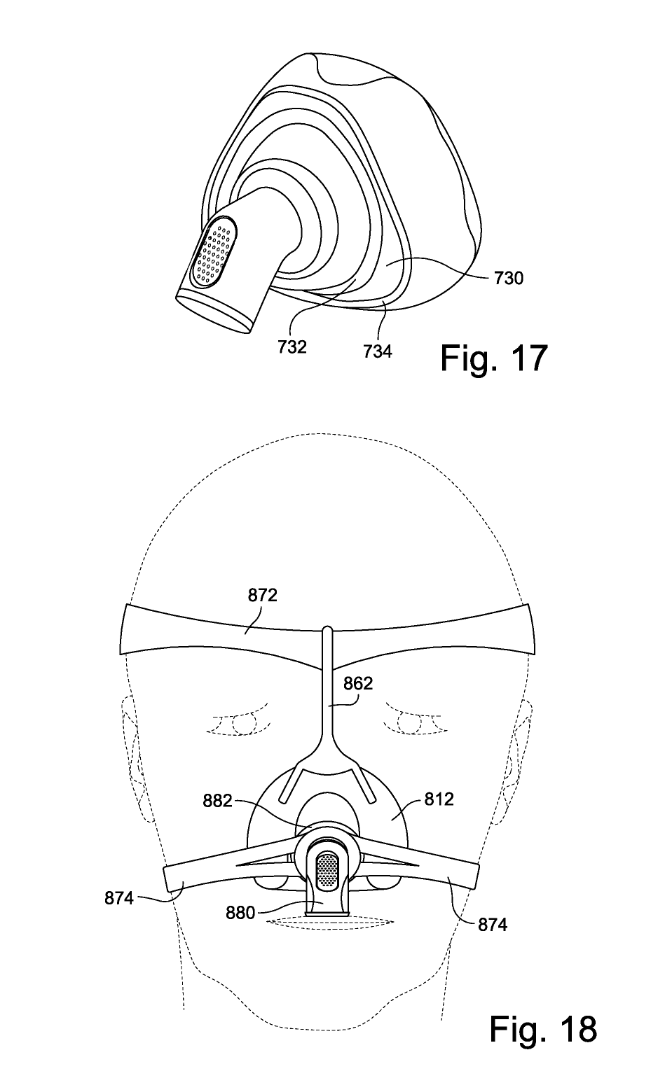

Also, the walls of the bladder may have different thicknesses with respect to one another, e.g., to control buckling. For example, FIG. 17 shows a cushion in which the inner wall 732 of the bladder 730 is thicker than the outer wall 734 thereof. It should be appreciated that the thicknesses may be different in different regions of the cushion.

A gate or hole 35, as shown in FIG. 1-8 is provided into the bladder to allow injection of the gel or super soft solid into the bladder. The gate may be covered (e.g., by a cap, by silicone injected over the gate, etc.) to enclose and retain the gel or super soft solid within the bladder.

In an alternative example, as shown in FIG. 4, the undercushion 20 may be spaced from the bladder 30, i.e., undercushion does not form a portion of bladder. In this arrangement, the cushion provides the sealing benefits of a dual-wall cushion as well the tactility and comfort of a fillable bladder. Also, such arrangement may help to control buckling and the force-deflection relationship.

### 2.4 Gel or Super Soft Solid in Selected Regions

The sealing arrangement includes bladders for fillable material (e.g., gel or super soft solid) in selected or targeted regions of the sealing arrangement to enhance comfort, support, fit range, and/or cost. Preferably, gel or super soft solid engages with the patient's face where a seal is formed due to compressive forces from the mask system in use. In the illustrated example, the gel or super soft solid bladder is only provided along the lower cheek and corner of nose regions of the cushion, i.e., the gel or super soft solid bladder is not provided about the entire perimeter of the cushion.

However, other arrangements of the undercushion and bladders are possible. For example, rather than an undercushion provided in all regions except the nasal bridge region as described above, the undercushion may be provided in three sections, i.e., undercushion at either side of the nose and a separate undercushion that extends from the corners of the nose and over the top lip. In this example, the bladder may be provided at the corners of the nose and cheek regions.

In another example, separate undercushions may be provided in four sections, i.e., undercushion at the sides of the nose and the corners of the nose. In this embodiment, the bladder may be provided at the corners of the nose.

In these alternative examples, the cushion will decouple the sides of nose seal from corners of the nose seal. This arrangement may be beneficial because the deformation of the fillable material (e.g., gel or super soft solid) may impact the position of the sides of nose seal portion of the undercushion.

In an example, the nasal bridge region and/or side of nose region of the cushion may have a bladder or fillable undercushion. The gap or distance between the undercushion defining an upper wall of the bladder and the membrane may be greater than other regions, to allow the membrane more freedom to move and adapt to the patient's face and therefore a possibility of the cushion fitting a wider anthropometric range. For example, FIG. 10 shows a cross-section through a side of nose region of a cushion according to an example of the disclosed technology. As illustrated, the gap between the membrane 15 and the upper wall 20 of the bladder 30 is relatively large, e.g., 3-7 mm.

FIG. 11 shows a cross-section through a corner of nose region of a cushion according to an example of the disclosed technology. In this illustrated example, the gap between the membrane 15 and the upper wall 20 of the bladder 30 is relatively small (e.g., 0-2 mm) to ensure the membrane is pushed and supported in the corner of nose region of the patient in use.

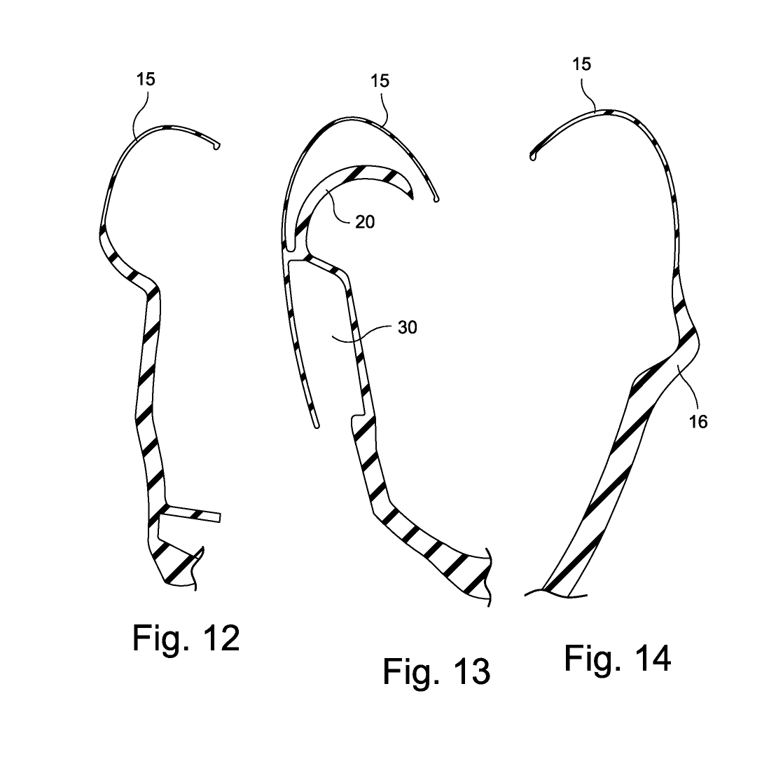

FIG. 12 shows a cross-section through an upper lip region of a cushion according to an example of the disclosed technology. In this illustrated example, the upper lip region has a single wall configuration (i.e., membrane 15 only with no undercushion). This arrangement may reduce force on the patient's top lip, which may be a sensitive area on some patients due to the underlying gum and tooth structure.

Like FIG. 4 described above, FIG. 13 shows an arrangement in which the undercushion 20 is spaced from the bladder 30, and therefore the membrane 15 and undercushion 20 provide a dual wall seal. Preferably this arrangement may be positioned at or proximal to the patient's cheek region. This arrangement provides the benefits of the dual wall seal and the added comfort of the gel or super soft solid bladder.

FIG. 14 shows a cross-section through a nasal bridge region of a cushion according to an example of the disclosed technology. In this example, a lower portion or base of the membrane 15 in the nasal bridge region may include a gusset or flexible portion 16 to assist in ensuring inwards collapse of the membrane in the nasal bridge region so as to prevent blow out of the flexible membrane and therefore potential leak into the patient's eyes in use. The thickness, outward extent or projection, and/or taper of the gusset may be adjusted to control flexibility or collapse of the membrane.

In an example, one or more walls of the bladder may be supported by localized regions of thickening, ribs, or a co-molded support portion like a clip, e.g., to prevent buckling. Such added wall support may be provided in one or more selected regions of the cushion.

In an example, the gel or super soft solid may leach or gradually penetrate the walls of the bladder. This leaching may be advantageously utilized as a moisturizer, aromatherapy, and/or end of life indicator (e.g., absence of scent/moisturizer from cushion indicates replacement is due). In an example, the gel and/or walls of the bladder may be suitably configured to control leach, e.g., control leach in one or more selected regions of the cushion.

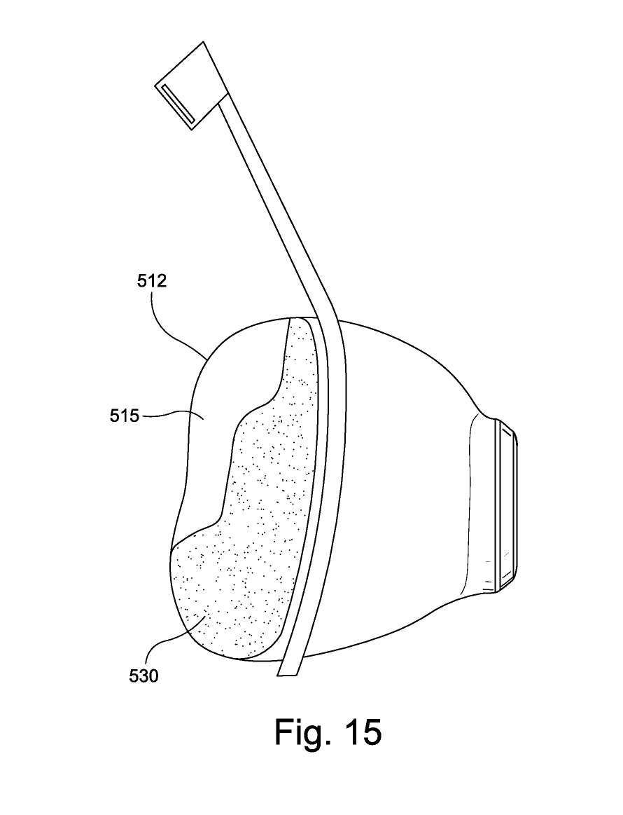

In an example, a dual wall seal (e.g., membrane and undercushion creating a tension seal) may be provided in the nasal bridge region, side of nose region, cheek regions, and/or top lip region of the cushion. Alternatively, the undercushion may define a portion of the gel or super soft solid bladder. In an example, the corner of nose region of the cushion may have only one wall, which may be a continuation of the undercushion. That is, the corner of nose region may only include a bladder or fillable undercushion. The corner of nose region of the cushion may be difficult to fit or seal due to the crease in the corner of the patient's nose, and the membrane can become wrinkled thereby creating a leak path. Accordingly, the membrane may be removed and the gel bladder may seal directly on the patient's face in the corner of nose region. In this example, a stand alone membrane is not present in all regions of the cushion. FIG. 15 illustrates a mask system including a cushion 512 according to an example of the disclosed technology. As illustrated, the membrane 515 is not present in the corner of nose region so that the bladder 530 directly seals on the patient's face in use. Also, the gap between the membrane 512 and the bladder may vary in different regions, e.g., gap is larger in nasal bridge and side of nose regions than cheek regions.

That is, the cushion may include a sealing membrane, a support layer, e.g., a fillable bladder filled with a gel or super soft solid, to support the sealing membrane, and a composite sealing portion including the sealing membrane and at least a portion of the support layer. The membrane may removed in at least one region of the cushion, e.g., the corner of nose region, such that the support layer is adapted to seal directly on the patient's face where the membrane is not present. The composite sealing portion may include a membrane part and a non-membrane part. The non-membrane part may be the support layer in which case the support layer has a dual function to support the membrane in at least one region, and to form a seal structure for sealing with the patient's skin. The membrane part and the support layer may include a transition region, or they may be substantially co-planar or otherwise flush or substantially seamless.

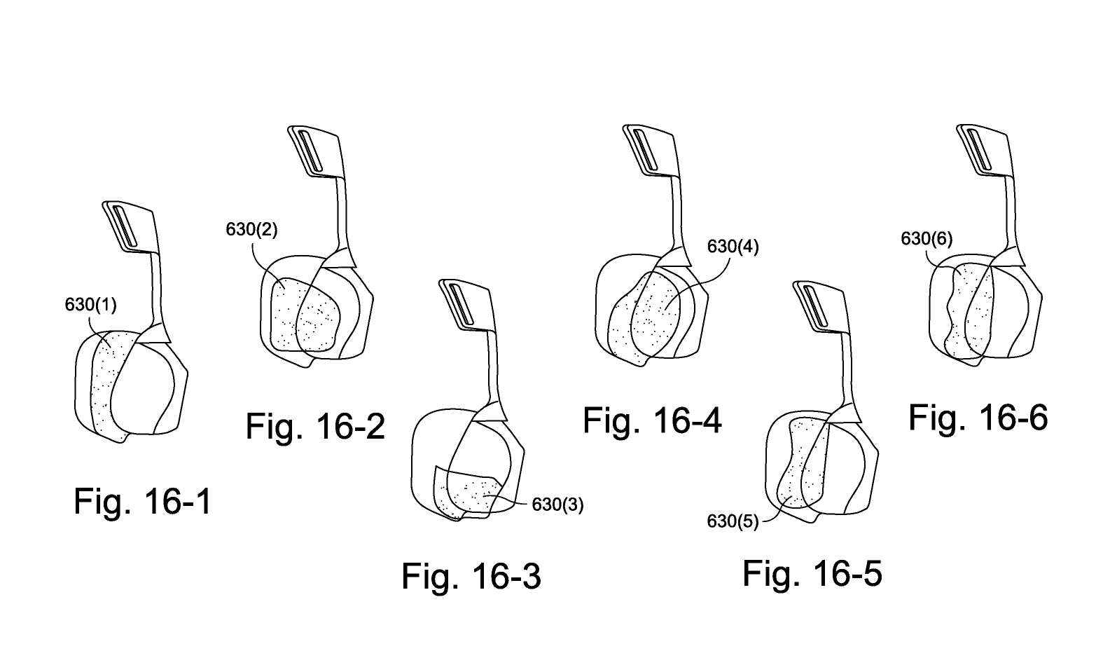

FIGS. 16-1 to 16-6 show cushions with bladder locations according to alternative examples of the disclosed technology. In FIG. 16-1, the bladder 630(1) is provided around the entire perimeter of the cushion. In FIG. 16-2, the bladder 630(2) is only provided in the side of nose and cheek regions of the cushion to assist in the lateral seal against the sides of the patient's nose in use. In this example, the bladder may allow for greater conformability of the cushion so as to fit high nose bridges as well as flat nose bridges. In FIG. 16-3, the bladder 630(3) is only provided in the corner of nose region of the cushion to aid in the compression seal in this region while ensuring comfort and a greater fit range due to the flexibility of the bladder. In FIG. 16-4, the bladder 630(4) is configured such that more bladder is provided in the corner of nose region than the side of the nose regions so as to create a substantially constant force profile. That is, the force exerted on the patient's face from the cushion is the substantially the same around the perimeter of the cushion. More force tends to be exerted in the corner of the nose, hence more bladder and hence gel or super soft solid is positioned in this region to absorb and distribute at least some of this force. In FIG. 16-5, the bladder 630(5) is configured such that more bladder is provided in the nasal bridge region so that the cushion can fit high nose bridges (where the gel will deform to suit) or low nose bridges (where the gel will support the membrane on the patient's nose bridge). FIG. 16-6 shows a bladder 630(6) with a relatively random pattern. It should be appreciated that the bladder of the cushion may include other suitable configurations or arrangements, and such configurations may be tailored to a particular patient.

### 2.4.1 Comfort

Compression of the cushion on the patient's face in use may result in discomfort, however there are localized regions of the patient's face where such a seal may be acceptable. Investigation has shown this is primarily in the cheeks and corners of the nose. In order to enhance comfort in these regions, gel or super soft solid Tillable bladders are provided to the cushion in the regions of the lower cheek and corner of nose. Since gel or super soft solid is flexible, it is possible for these materials to spread compressive forces over a wider region.

### 2.4.2 Anchor

Also, the compression force may be utilized to anchor, stabilize or support the cushion in position. Investigation has also shown that anchoring the cushion at the corners of the nose is ideal because the firmer tissue in this region enhances stability. It is also possible to anchor the mask on the softer tissues at the cheeks as this area is relatively insensitive to contact pressure.

### 2.4.3 Fit Range

Since some gels or super soft solids are more flexible than rubber components, they are better able to adapt to the geometry of patient's face in use. Some patients may have deep creases at the corners of the nose and cheeks or may have relatively flat cheeks or corners of the nose, and thus greater flexibility of the cushion is required in these regions so as to fit a wider range of patients.

### 2.5 Over-Tighten Feature

In an example, the gel or super soft solid bladder may include a bulge or thickened region where the gel or super soft solid may compress outwards and over the frame in the event that the headgear is over-tightened so that the patient's face is protected from contact with the substantially rigid mask frame. Comfort may be enhanced by this arrangement.

For example, FIGS. 2-1 and 2-2 illustrate alternative examples of a gel or super soft solid bladder. As illustrated, a base or lower portion of each bladder includes a bulge or thickened region 36 to protect against over-tightening. In addition, the bladder includes a lip 31 that is curved inwardly towards the breathing chamber to encourage rolling, and bladder walls 32, 34 that are shaped and contoured to encourage controlled buckling and protect against over-tightening. In use, the gel or super soft solid bladder diverges towards the base and provides a bulge at the base to allow the gel or super soft solid to spread outwards under load and hence distribute pressure.

### 2.6 Controlled Buckling

As best shown in FIGS. 1-12 and 1-13, the undercushion 20 interfaces with the inner wall 32 to form the bladder 30. Such interface may at least partially determine how the bladder wall buckles during deflection in use. Controlling how the bladder wall buckles helps prevent occlusion of the nares and may also ensure comfort of the mask in use.

Although the walls 32, 34 of the bladder 30 are relatively deep as shown in FIG. 1-15, the active part of the wall with gel or super soft solid is shortened by interaction of the cushion with the frame 50. The frame separates the gel that interfaces with the patient's face with the remaining gel, i.e., the frame defines an axis A that separates a functional portion of the bladder (represented by arrow 30(1)) from a tactile/aesthetic window portion (represented by arrow 30(2)) of the bladder. In an example, the walls of the bladder may be shortened, but sufficiently long to control buckling in use.

The tactile/aesthetic window portion 30(2) of the bladder also serves as gel overflow area, i.e., a place into which the gel may displace as described above with respect to the over-tighten feature. This reduces the feeling of sudden rigidity during maximum compression of the bladder.

FIG. 3-1 shows a bladder in which the inner wall 32 intersects the undercushion 20 near its edge. At the point of intersection, the inner wall 32 and the undercushion 20 form a generally T-shaped cross-section which is inherently rigid but not generally stable in deflection. As the undercushion 20 is loaded in use, the inner wall 32 must buckle and the interface between the undercushion and inner wall is such that the buckling is not biased to any particular direction thus controlled collapse is not possible.

Uncontrolled buckling can lead to inconsistent behavior when masks are used on multiple patients. Furthermore, certain geometries may lead to cushion features that must deflect “over-center” causing a flicking or clicking motion rather than smooth, controlled deflection. FIGS. 3-2 to 3-4 show exemplary examples that are structured to provide more controlled buckling of the bladder wall in use.

As shown in FIGS. 3-2 to 3-4, a curved wall portion 133 (FIG. 3-2) or a slanted wall portion 233, 333 (FIGS. 3-3 and 3-4) may be provided to the inner wall 32 to intersect with the undercushion 20. In FIG. 3-4, the slanted wall 333 intersects the undercushion 20 at a position that is relatively closer (i.e., compared to that of the wall portions 133, 233) to the outer wall 34 and the inner fixed end of the undercushion 20. Such curved or slanted wall portion provides an intersecting feature that may bias the bladder wall to collapse in a controlled manner. However, it should be appreciated that other wall configurations are possible, e.g., different permutations of the curved or slanted wall portion.

FIG. 3-4 shows an example of the slanted wall portion 333 being deflected in use. As illustrated, the membrane 15, undercushion 20, outer bladder wall 34, and wall portion 333 all intersect with one another at a relatively stiff region 38 (e.g., area enclosed by dashed circle) of the cushion. As the undercushion 20 is deflected in use (as represented by hatched and unhatched sections of the undercushion), the wall portion 333 deflects or bends inwardly into the bladder chamber (as represented by hatched and unhatched sections of the wall portion) and this deflection mode avoids “over center” bending or flicking, by biasing toward a smooth deflection mode. It should be appreciated that the curved wall portion 133 and slanted wall portion 233 will move or bend further inwardly into the bladder chamber in a similar manner.

In an example, as shown in FIG. 3-4, the portion of the molding tool T (indicated in dashed lines) that forms the meeting point p between the membrane 15 and undercushion 20 may include a rounded end with a radius r1 so as to provide the meeting point p with a rounded configuration with a radius r2 similar to or the same as r1. Such rounded configuration may prevent tearing between the membrane and undercushion and allow the use of a rounded molding tool which prevents damage to the cushion during manufacturing, e.g., no sharp edges or points on molding tool.

In each example, the curved and slanted wall portions 133, 233, 333 are structured to deflect into the bladder chamber without passing or “clicking” over a rigid midway position. For example, in contrast to the curved wall portion 133 including a concave configuration that curves into the bladder chamber, a curved wall portion including a convex configuration that curves away from the bladder chamber must deflect over-center causing a flicking or clicking motion, i.e., convex curved wall portion must flick or click past the center or halfway point in which the wall is generally flat. Thus, the curved and slanted wall portions 133, 233, 333 provide geometries that provide a smooth, controlled deflection in use.

FIGS. 3-5(1) and 3-5(2) show another example of a bladder or gel or super soft solid tillable undercushion that is structured to provide a more controlled collapse in use. As illustrated, the walls and lip of the bladder are shaped, contoured, and/or profiled such that the cushion deflects in a particular way to prevent or mitigate occlusion of the patient's nose/nares, prevent the patient's face from contacting the mask's relatively rigid frame, and/or ensure, through prevention of patient/frame contact, that the system's sensitivity to headgear tension is reduced (i.e., soft material always sits between the patient and the frame).

In the illustrated example, the cushion includes a membrane 15 and an undercushion 20 that defines a tillable bladder 30 adapted to be filled with a soft fellable material (e.g., tactile material such as gel or super soft solid or ultra-soft solid). The cushion is structured to be attached to a relatively rigid frame 50. In the illustrated example, as shown in FIG. 3-5(2), the cushion is structured to deflect when a load L is applied such that the cushion material always obscures the frame 50 and the bladder 30. When no load is applied, as shown in FIG. 3-5(1), the distance between the outermost edge of the frame and the outermost edge of the cushion is the distance d1. However, when a load is applied, as shown in FIG. 3-5(2), the distance between the outermost edge of the frame and the outermost edge of the cushion becomes the distance d2. When a load is applied, the cushion deflects such that the distance d2 is greater than the distance d1, thereby preventing the patient from contacting the frame 50. Furthermore, the undercushion shape promotes a deflection mode that avoids occlusion of nares by controlled collapse by outwards biasing. The frame 50 provides a brace or fulcrum like structure about which the cushion may pivot, bend, and/or flex. The frame may serve as a pivot point for the bladder to fold over to avoid bottoming out directly onto the frame. The cushion provides sufficient flexibility in the side walls of the bladder, tending to roll out rather directly tighten to face.

### 2.7 Gel or Super Soft Solid

The bladder may be filled with a single fellable material (e.g., gel or super soft solid) or the bladder may be filled with two or more fillable materials (e.g., gels or super soft solids) or layers of tillable materials having different properties (e.g., gels or super soft solids of different hardness).

In an example, the tillable material is a silicone gel or super soft solid. However, other gels, super soft solids, gel-like materials, or super soft solid-like materials may be used.

The hardness of the gel or super soft solid may be selected to improve comfort and/or sealing ability for example.

### 2.8 Overflow Region

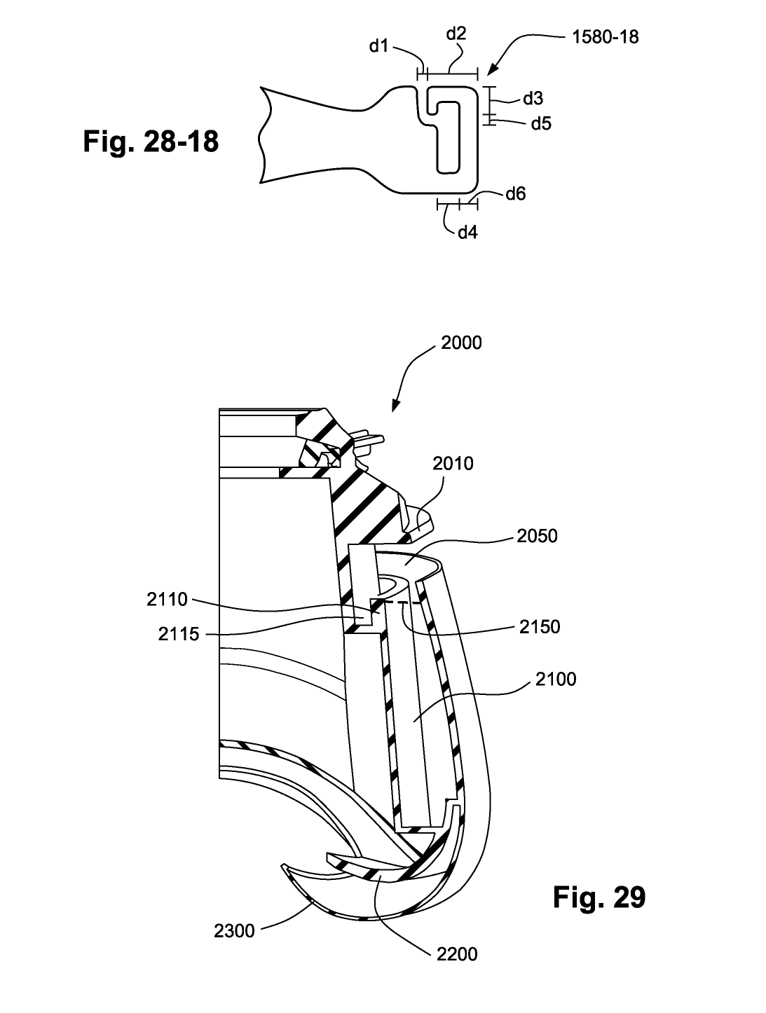

In an example, the cushion may include a bladder having an overflow region. FIG. 29 shows a cushion 2000 in cross section, including a membrane 2300 adapted to interface and seal on the patient's face, an undercushion 2200 adapted to support the membrane in position, and a bladder 2100 adapted to be filled with a conformable material such as a gel or super soft solid. An injection opening 2050 permits entry of a nozzle to fill the bladder 2100 with the conformable material and a cap 2010 covers the injection opening 2050.

The cushion 2000 further includes an overflow region 2115 adjacent the bladder 2100 and separated from the bladder 2100 by a bladder dividing wall 2110.

When the conformable material (not shown) is injected via the injection opening 2050 into the bladder 2100, a pre-set amount of conformable material is dispensed. If the volume of the bladder is different from the specified amount of conformable material some of the conformable material may exceed a maximum fill level (indicated at line 2150). The volume of the bladder may be different from the specified amount if the shape of the bladder is somehow changed after molding (e.g. if de-molded and placed in a jig or nest, the bladder was molded out of specification or the bladder was molded at an extreme end of its tolerance range). The maximum fill level may depend on the maximum height of the dividing wall 2110.

If the conformable material exceeds the maximum fill level 2150 , the conformable material may flow into the over flow region 2115. Accordingly, the total height of the conformable material may never exceed the maximum fill level. This arrangement ensures the maximum fill height of the bladder is constant, and thus enables a rigid component (e.g., a frame) to interface with the filled bladder at the same location on all cushions.

### 3. Frame

FIG. 7 illustrates an example of a frame 250 to maintain, stabilize and/or support the cushion. As illustrated, the frame 250 includes a main body 252 providing an aperture 254 to retain or otherwise engage the cushion 12. A forehead support 260 extends from the top of the main body. The forehead support includes an elongated arm 262 and an upper cross portion 264 providing slots 265 adapted to receive upper headgear straps of headgear. Hook-shaped lower headgear connectors 268 are provided to respective sides of the main body and adapted to engage lower headgear straps of headgear. Side engagement tabs 266 extend from the main body and are positioned to interface and support the cushion. In an example, the tabs may support the cushion so that it is supported in sealing engagement with the sides of the patient's nose or cheek region. Also, the tabs may support the cushion to prevent collapse or blowout of the cushion away from the patient's face in use.

In an example, the frame may assist in retaining and positioning the cushion as well as capping the bladder. For example, the bladder may be open along the non-face-contacting side of the cushion (i.e., the one or more openings, gates or holes into the bladder to allow injection of the gel or super soft solid may be exposed), and the frame may abut these one or more openings to close them off. In an example, the frame may have one or more lugs or protrusions adapted to engage with respective openings of the bladder, which may further aid alignment of the cushion with respect to the frame.

FIGS. 8-1 to 8-6 illustrate a frame 350 according to another example. As illustrated, the frame 350 includes a single, one piece construction (e.g., molded of nylon, polypropylene, polycarbonate) with a main body 352 and a forehead support 360 extending from the top of the main body. The main body 352 includes an aperture 354 to receive, retain or otherwise engage the cushion. The forehead support includes an elongated arm 362 and cross arms 364 each providing a slot 365 adapted to receive a respective upper strap of headgear. Lower headgear connectors are provided to respective sides of the main body and adapted to engage lower straps of headgear. Each lower headgear connector includes an elongated arm 368 and a slot or hook 369 adapted to receive the respective lower headgear strap.

As best shown in FIGS. 8-2 to 8-4, the rear portion of the frame 350 includes a lug 355 that extends around the aperture 354. The lug 355 is structured to interface with the geometry on the cushion for alignment and glide. That is, the lug 355 includes a curvature or scoop-shape (e.g., see FIG. 8-4) that is structured to substantially match or closely follow the geometry of the non-face-contacting or frame side of the cushion. In use, the non-face-contacting side of the cushion would be positioned adjacent this lug or scoop (i.e., cushion touching but not coupled to the lug). The cushion would be able to rock (as indicated by the arrows in FIG. 8-4) up and down along the lug relative to the frame in use, thereby adjusting the position of the cushion relative to the frame and allow the seal to accommodate for variation in patient's facial features (e.g., achieving a similar affect as an adjustable forehead support).

FIGS. 8-5 and 8-6 show engagement of the cushion 412 with the frame 350.

FIGS. 9-1 to 9-4 illustrate a frame 450 according to another example. The frame 450 is substantially similar to the frame 350 described above. In contrast, the frame 450 includes an aperture 468(1) in the arm 468 of each lower headgear connector and an aperture 462(1) in the elongated arm 462 of the forehead support. The apertures 462(1), 468(1) are provided to reduce visual bulk, reduce cost, and/or enhance flexibility of the respective arm 462, 468.

FIGS. 9-2 to 9-4 show engagement of the cushion 412 with the frame 450, and support of the mask system by headgear on the patient's head.

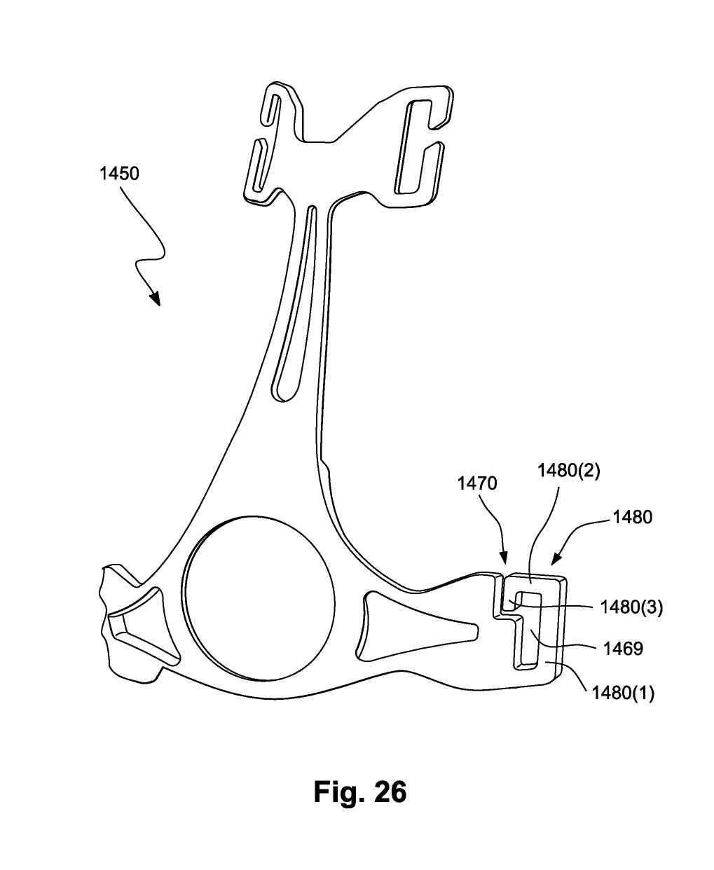

FIG. 26 illustrates a frame 1450 according to another example. The frame 1450 is substantially similar to the frame 450 described above. In contrast, the frame 1450 includes a hook 1480. The advantage of a hook having the illustrated or similar arrangement is that the strap is easier to insert than it is to remove, which functions to ensure accidental disassembly is avoided. The hook 1480 includes a hook arm 1480(1), a retaining portion 1480(2) and a depending arm 1480(3) extending from the retaining portion. The hook 1480 forms a retaining slot 1469 for accommodating a strap in use. When a strap is inserted into the retaining slot 1469, via a slot 1470, the retaining portion 1480(2) and the depending arm 1480(3) function to help retain the strap in the retaining slot 1469. As shown, the slot 1470 is parallel to the retaining slot 1469, and a perpendicular channel extends between the slots 1469, 1470. This arrangement is optimum for ensuring that accidental disassembly is prevented. Since it is difficult for the strap to move from the slot 1469 into a perpendicular position accidentally, assistance is required to maneuver the strap through the channel to the slot 1470. The depending arm 1480(3) further aids in retaining a strap in the slot 1469 thereby further preventing accidental disassembly.

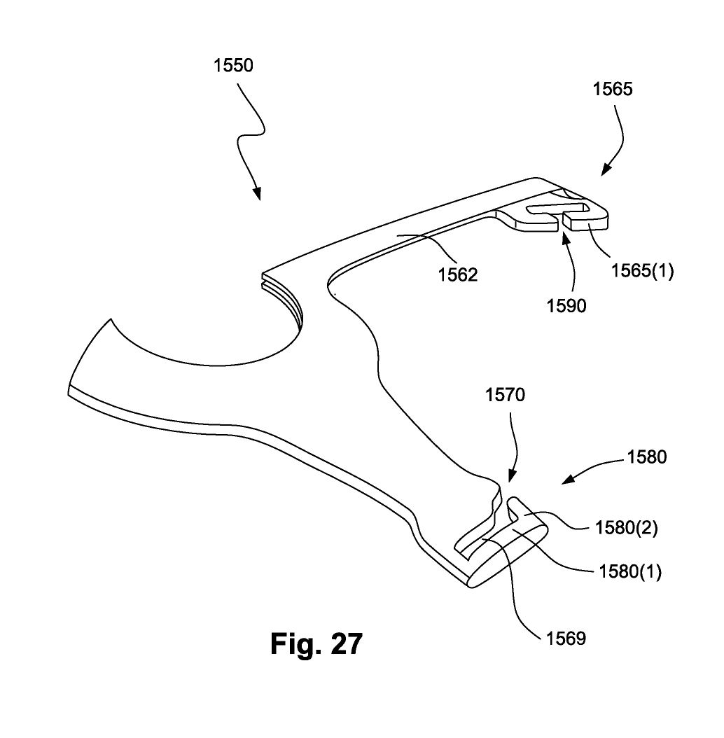

FIG. 27 illustrates a frame 1550 according to another example. The frame 1550 is substantially similar to the frame 350 described above. In contrast, the frame 1550 includes a hook 1580. The hook 1580 is similar to the hook 1480 described above, and includes a hook arm 1580(1) and a retaining portion 1580(2). The hook 1580 forms retaining slot 1569. However, in contrast to the hook 1480, the retaining portion 1580(2) does not include a depending arm and as such, may make it easier to assemble a strap into the slot 1569. Also, the slot 1570 is inclined with respect to the slot 1569 thereby guiding a strap more readily in and out of the retaining slot 1569.

In further contrast to the frame 350, the frame 1550 also eliminates the cross arm 364 and has a hook 1565 connected directly to the elongated arm 1562. The hook 1565 includes a pair of retaining portions 1565(1) separated by a slot 1590.

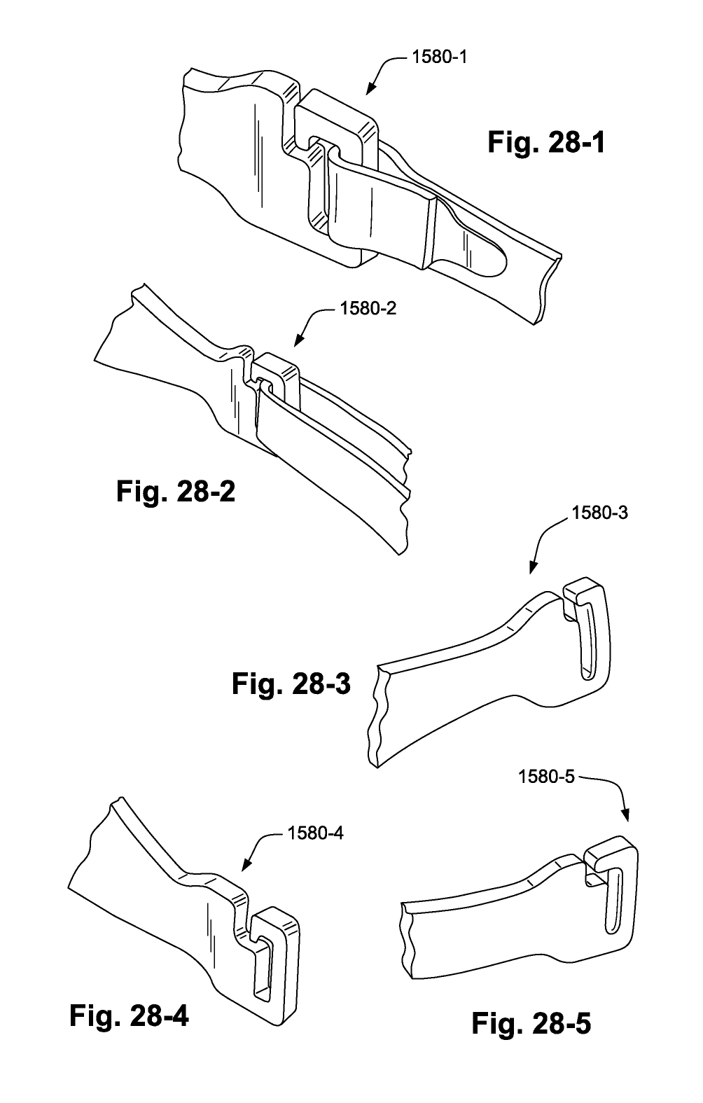

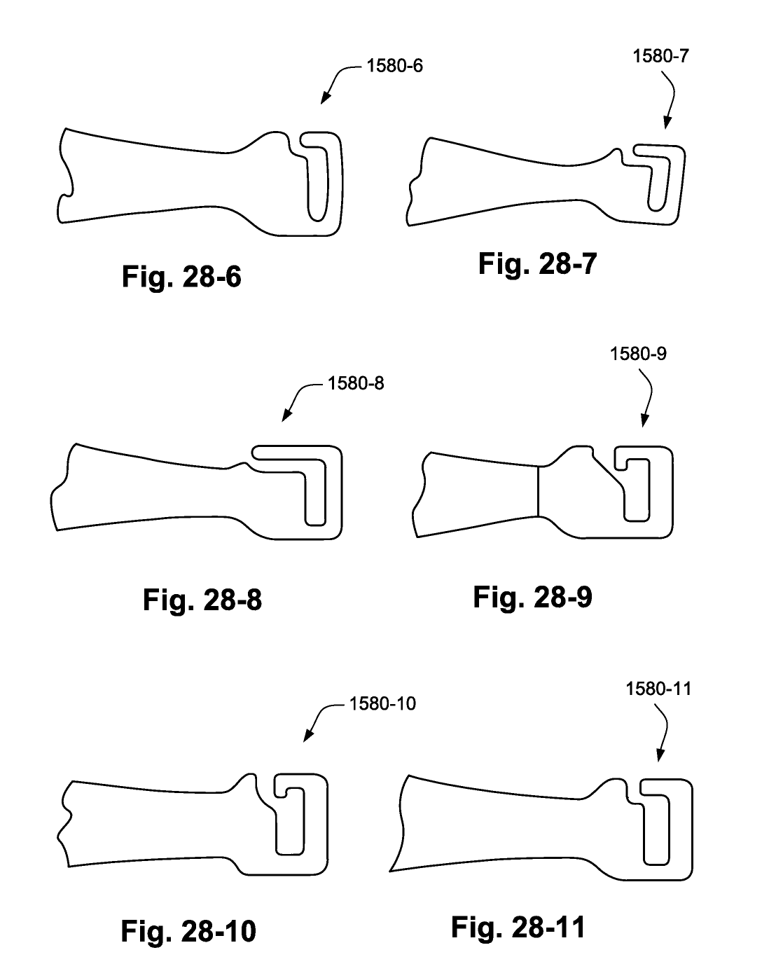

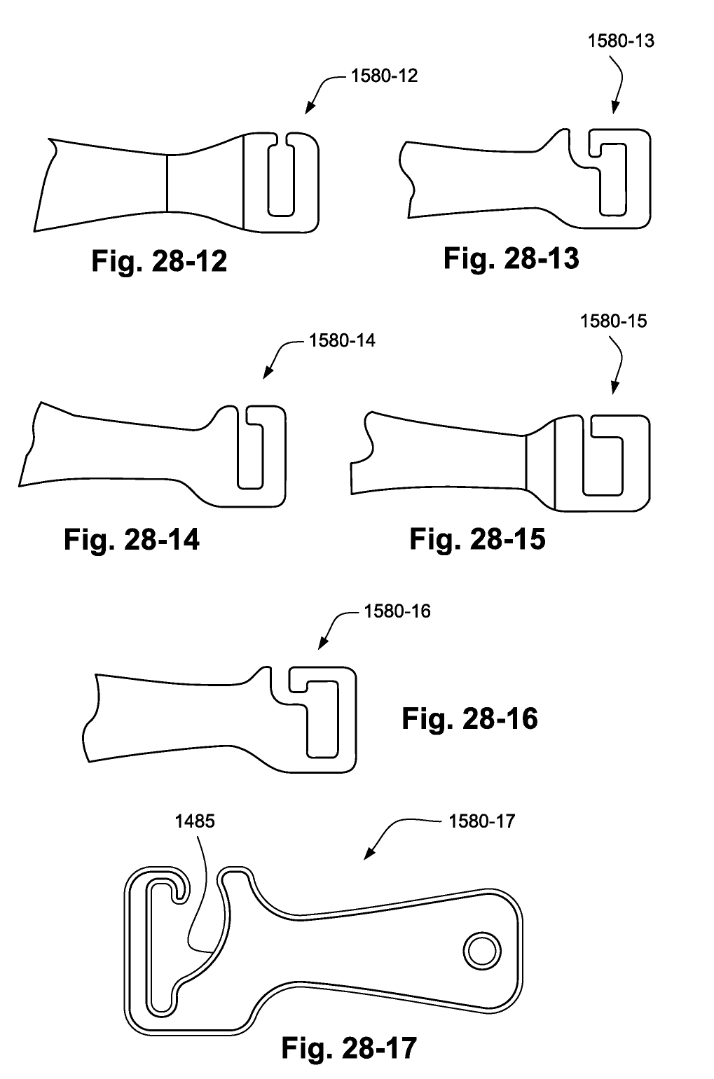

FIGS. 28-1 to 28-18 show various configurations of a hook 1580-1 to 1580-18. These hooks are similar to the hook 1580 described above. It is noted that the hooks may include a curved surface 1485, as shown in FIG. 28-17 to facilitate insertion of the strap into the retaining slot. However, using FIG. 28-18 as an example, the distances d1, d2, d3, d4, d5 and d6 can be varied to construct numerous hook configurations. One skilled in the art will appreciate that the various hook configurations described as usable with lower headgear straps can also be used with upper headgear straps, and vice versa.

For example, d1 may represent the width of the opening of the slot 1570. In an example, d1 may be relatively narrow as in FIGS. 28-8, 28-14, 28-15, 28-18 to make it difficult to accidentally disassemble the strap from the hook. Alternatively, d1 may be widened such as in FIG. 28-7, 28-10, 28-13, 28-16 to make it easier to assemble the strap to the hook.

In a further example, d2 may represent the width of a retaining portion such as retaining portion 1480(2). In an example, d2 may be greater than the width of the strap in order to better retain the strap in the slot, such as in FIGS. 28-13 and 28-15. Alternatively, d2 may be relatively similar in width to the strap thereby making assembly easier, such as in FIGS. 28-12 and 28-14.

In a further example, d3 may represent the length of a depending arm such as depending arm 1480(3). The length of d3 may be relatively small, see for example FIG. 28-16, or relatively long (FIG. 28-13). A relatively longer depending arm makes it easier to retain the strap. A relatively shorter depending arm may minimize the difficulty to disengage the strap from the slot.

In a further example, d4 may represent the width of the retaining slot 1469. In an example, d4 is wider than the width of a strap, as shown in FIG. 28-15, to make it easier for a patient to disassemble the strap from the frame, as the added width of the slot may make it possible for the patient to bend the strap around into the the slot 1470, 1570. Alternatively, d4 may be relatively similar to the width of the strap to ensure that the strap maintains its desired orientation.

In addition, d5 may represent the height of the gap or channel between the slot 1470, 1570 and the slot 1469, 1569. The greater the height, the easier removal of the strap becomes; however an increased height may also increase the risk of accidental disassembly. Furthermore, d5 may also represent whether the gap is perpendicular to the slot 1469, 1569, as in FIGS. 28-6 and 28-7, or sloped, as in FIGS. 28-9 and 28-10. The strap may be easier to slide into the slot and easier to remove from the hook if the gap represented at d5 is sloped.

Finally, d6 may represent the width of the hook arm 1480(1), 1580(1). Preferably, d6 is at least 3 mm to ensure that the hook, is robust and less than 15 mm to avoid discomfort to the patient.

### 4. Headgear

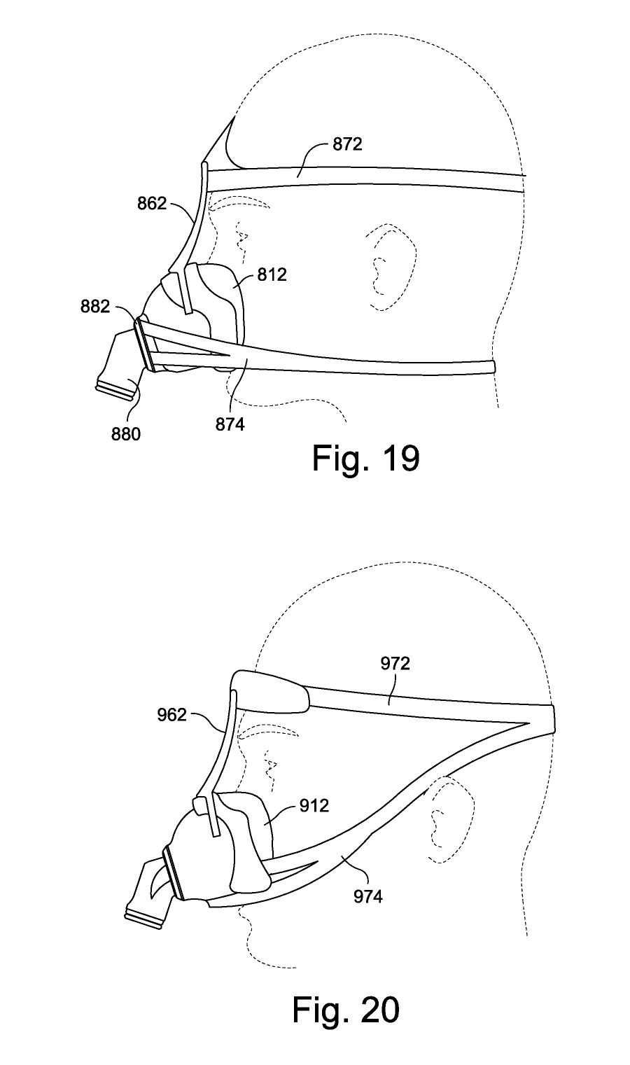

FIGS. 18 to 22 show mask systems with headgear arrangements according to alternative examples of the disclosed technology. In FIGS. 18 and 19, an upper headgear strap 872 of headgear is engaged with a forehead support or forehead support bar 862 provided to the cushion 812, and lower headgear straps 874 are engaged with the non-face-contacting side of the cushion adjacent to the aperture supporting the elbow 880, e.g., lower headgear straps engaged with seal ring 882 supporting elbow 880 within cushion 812. As illustrated, the end of each lower headgear strap may be bifurcated for attachment to the cushion. The lower headgear straps may be attached to the cushion or other component of the frame by an interference fit, e.g., a lug on the headgear may be received by an aperture on the cushion or vice versa. Alternatively, the lower headgear connectors may wrap around the elbow or frame. Alternatively, lower headgear straps may connect to an intermediate connector such as a headgear clip, with the intermediate connector being received by the cushion and/or frame. In a further alternative, lower headgear straps may be formed integrally with the cushion.

In FIG. 20, upper headgear straps 972 of headgear are engaged with a forehead support 962 provided to the cushion 912, and lower headgear straps 974 are engaged with lower sides of the cushion 912. As illustrated, the end of each lower headgear strap 974 may be bifurcated to provide two attachment points to the respective lower side of the cushion. The lower headgear straps may be attached to the cushion or other component of the frame by an interference fit, e.g., a lug on the headgear may be received by an aperture on the cushion or vice versa. Alternatively, the lower headgear connectors may wrap around the elbow or frame. Alternatively, lower headgear straps may connect to an intermediate connector such as a headgear clip, with the intermediate connector being received by the cushion and/or frame. Alternatively, the upper bifurcated portion of the lower headgear strap may attach, for example by an interference fit with the cushion, and the lower bifurcated portion of the lower headgear strap may be continuous with or joined to the respective lower bifurcated portion on the opposing lower headgear strap. In a further alternative, lower headgear straps may be formed integrally with the cushion.

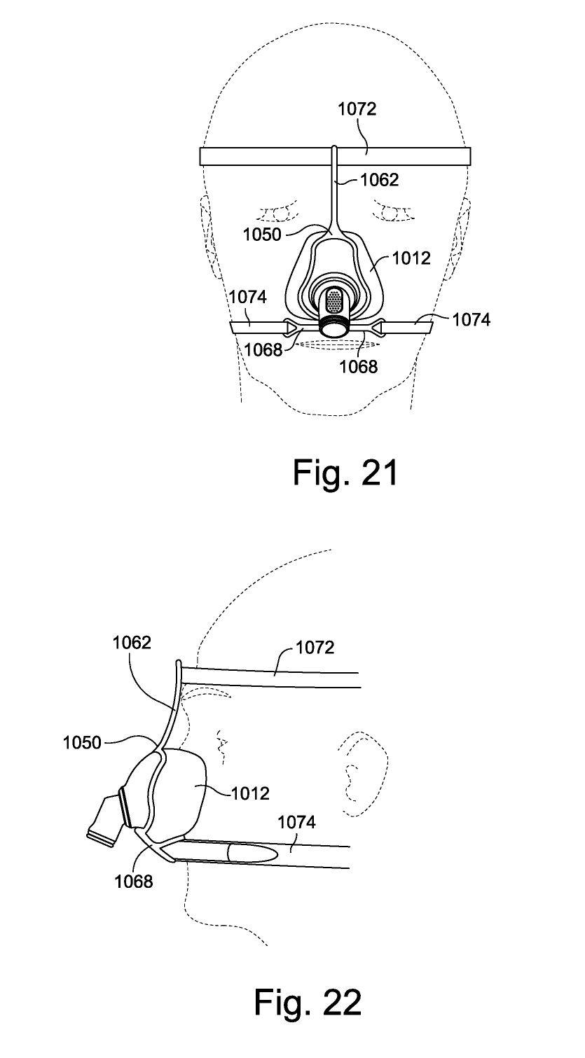

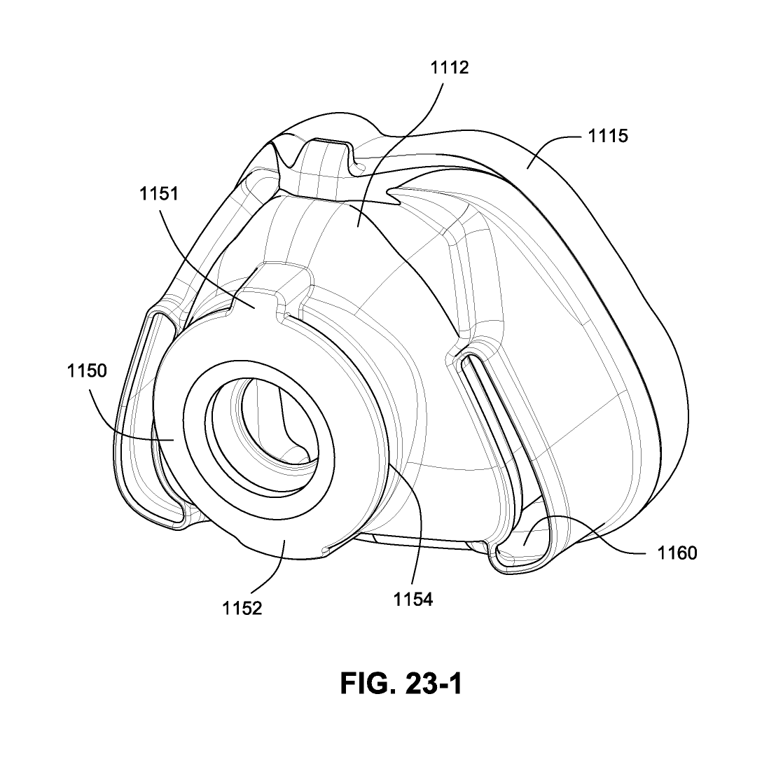

In FIGS. 21-22, the cushion 1012 is supported by a wire-type frame 1050 with an open construction structured to wrap around the cushion. The frame 1050 provides a forehead support or forehead support bar 1062 for attaching an upper headgear strap 1072 and lower headgear connectors 1068 for attaching lower headgear straps 1074. Upper headgear straps 1072 may be received in one or more apertures in the forehead support bar 1062 and attach by hook and loop material as is known in the art. Alternatively, upper headgear strap 1072 may be a continuous loop around the circumference of the patient's head. Lower headgear straps 1074 may be received in one or more apertures in the lower headgear connectors 1068.

While the technology has been described in connection with several examples, it is to be understood that the technology is not to be limited to the disclosed examples, but on the contrary, is intended to cover various modifications and equivalent arrangements included within the spirit and scope of the technology. Also, the various examples described above may be implemented in conjunction with other examples, e.g., one or more aspects of one example may be combined with aspects of another example to realize yet other examples. Further, each independent feature or component of any given assembly may constitute an additional example. In addition, while the technology has particular application to patients who suffer from OSA, it is to be appreciated that patients who suffer from other illnesses (e.g., congestive heart failure, diabetes, morbid obesity, stroke, bariatric surgery, etc.) can derive benefit from the above teachings. Moreover, the above teachings have applicability with patients and non-patients alike in non-medical applications.

What is claimed is:

1. A mask system, comprising:
a frame; and
a cushion provided to the frame,
wherein the frame provides a brace or fulcrum structure about which the cushion may pivot, bend, and/or flex.

2. A mask system according to claim 1, wherein one or more portions of the cushion include a fillable bladder filled with gel or super soft solid.

3. A mask system according to claim 2, wherein the frame provides a pivot point for the bladder to fold over in use and avoid bottoming out onto the frame.

4. A sealing arrangement for a mask system, comprising:
a cushion molded in a single piece from an elastic material,
the cushion including a sealing membrane adapted to form a seal with a patient's face, an undercushion, and a fillable bladder filled with a gel or super soft solid,
the undercushion and fillable bladder provided in one or more selected regions of the cushion,
wherein the undercushion is provided in all regions of the cushion except a nasal bridge region of the cushion and the fillable bladder is provided in lower cheek and corner of nose regions of the cushion.

5. A mask system, comprising:
a frame including a generally open construction; and
a cushion including a breathing chamber and adapted to form a seal with a patient's face, the cushion being supported by the frame such that an exterior surface of the cushion engages an interior surface of the frame,
one or more portions of the cushion including a fillable bladder filled with a gel or a super soft solid.

6. A mask system according to claim 5, wherein the cushion includes a sealing membrane adapted to form a seal with the patient's face and further includes an undercushion in one or more selected regions of the cushion.

7. A mask system according to claim 6, wherein the undercushion is provided in all regions of the cushion except a nasal bridge region of the cushion.

8. A mask system according to claim 6, wherein the undercushion is provided in all regions of the cushion except a nasal bridge region of the cushion and an upper lip region of the cushion.

9. A mask system according to claim 7, wherein a lower portion of the membrane in at least the nasal bridge region of the cushion includes a gusset.

10. A mask system according to claim 6, wherein the undercushion defines an upper wail of the bladder.

11. A mask system according to claim 10, wherein the undercushion intersects with an inner wall of the bladder, and the inner wall includes a curved or slanted portion providing a feature to control buckling of the inner wall in use.

12. A mask system according to claim 11, wherein the undercushion includes a free end that overhangs the inner wall.

13. A mask system according to claim 6, wherein the undercushion is spaced from the bladder such that the sealing membrane and the undercushion provide a dual wall seal.

14. A mask system according to claim 6, wherein a gap between the sealing membrane and the undercushion varies in different regions of the cushion.

15. A mask system according to claim 5, wherein the fillable bladder is only provided in a lower cheek region of the cushion and a corner of nose region of the cushion.

16. A mask system according to claim 5, wherein the fillable bladder is only provided in side of nose, upper cheek, lower cheek, and corner of nose regions of the cushion.

17. A mask system according to claim 6, wherein the cushion includes only a fillable bladder in a corner of nose region of the cushion adapted to form a seal with the patient's face.

18. A mask system according to claim 5, wherein the frame includes a lug to interface with the cushion for alignment and glide.

19. A mask system according to claim 18, wherein the lug includes a curvature or scoop-shape that substantially matches a geometry of a non-face-contacting side of the cushion.

20. A mask system according to claim 5, wherein the cushion includes a sealing membrane adapted to form a seal with the patient's face, and a gap between the sealing membrane and an upper wall of the bladder is between about 0-2 mm.

21. A cushion for a mask system, comprising:
a sealing membrane adapted to form a seal with a patient's face;
a support layer adapted to support the sealing membrane; and
a composite sealing portion including the sealing membrane and at least a portion of the support layer,
wherein the sealing membrane is removed in at least one region of the cushion such that the support layer is adapted to seal directly on the patient's face.

22. A cushion according to claim 21, wherein the sealing membrane is removed in a corner of nose region of the cushion.

23. A cushion according to claim 21, wherein the support layer includes a tillable bladder filled with a gel or a super soft solid.

24. A cushion according to claim 23, wherein a gap between the sealing membrane and the bladder varies in different regions of the cushion.

25. A cushion according to claim 24, wherein the gap is larger in nasal bridge and side of nose regions of the cushion than in cheek regions of the cushion.

26. A cushion according to claim 21, further comprising a transition region between the sealing membrane and the support layer.

27. A mask system, comprising:
a frame; and
a cushion provided to the frame,
wherein the frame interfaces with the cushion to separate a functional or active portion of the cushion from a tactile or aesthetic portion of the cushion.

28. A mask system according to claim 27, wherein one or more portions of the cushion include a tillable bladder filled with a gel or a super soft solid.

29. A mask system according to claim 28, wherein the tactile or aesthetic portion of the cushion provides a gel overflow area for compressed gel from the functional or active portion of the cushion.

30. A cushion for a mask system, comprising:
a membrane adapted to form a seal with a patient's face; and
an undercushion adapted to support the membrane,
wherein the undercushion comprises a bladder portion structured to receive a conformable material, and the membrane is constructed and arranged to form in use a compression-type seal with the patient in a corner of nose region of the cushion, and the membrane is constructed and arranged to form in use a tension-type seal with the patient in a nasal bridge region of the cushion.

31. A cushion according to claim 30, wherein the undercushion is located at a side of nose region of the cushion.

32. A cushion according to claim 30, wherein the bladder portion is located at the corner of nose region of the cushion.

33. A cushion according to claim 30, wherein the conformable material is a gel or super soft solid.

34. A cushion for a mask system, comprising:
a bladder adapted to receive a conformable material and having a maximum fill level; and
an overflow region adjacent the bladder and adapted to receive any portion of the conformable material exceeding the maximum fill level.

35. The cushion of claim 34, further comprising a dividing wall positioned between the overflow region and the bladder.

36. The cushion of claim 34, further comprising an injection opening adjacent the overflow region and adapted to permit entry of a nozzle to fill the bladder with the conformable material.

37. The cushion of claim 36, further comprising a cap covering the injection opening.

38. The cushion of claim 34, further comprising a membrane adapted to form a seal with a patient's face,
wherein the bladder includes a portion forming an undercushion adapted to support the membrane.

39. A sealing arrangement for a mask system, comprising:
a cushion molded in a single piece from an elastic material,
the cushion including a sealing membrane adapted to form a seal with a patient's face, an undercushion, and a fillable bladder filled with a gel or super soft solid,
the undercushion and fillable bladder being provided in one or more selected regions of the cushion,
wherein the undercushion is provided in all regions of the cushion except a nasal bridge region, and the fillable bladder is only provided in side of nose, upper cheek, lower cheek and corner of nose regions of the cushion.

40. The sealing arrangement of claim 39, wherein the undercushion is spaced from the bladder such that the sealing membrane and the undercushion provide a dual wall seal.

41. The sealing arrangement of claim 39, wherein a depth of the fillable bladder varies along sides of the cushion.

42. A mask system, comprising:
a frame having an arm including a hook for receiving a strap; and
a cushion provided to the frame;
wherein the hook includes a retaining portion defining a first slot for accommodating the strap in use, and the frame includes a second slot adjacent the hook for inserting the strap into the first slot.

43. The mask system of claim 42, wherein the hook has a depending arm extending from the retaining portion to aid in retaining the strap in the first slot.
